(19) United States
(12) Patent Application Publication
(10) Pub. No.: US 2026/0218319 A1
(43) Pub. Date: Jul. 30, 2026

(54) COMPOSITIONS, KITS AND METHODS FOR DETECTION OF VIRAL SEQUENCES

(71) Applicant: LIFE TECHNOLOGIES CORPORATION, Carlsbad, CA (US)

(72) Inventors: Pius M. BRZOSKA, Woodside, CA (US); Elvis HUARCAYA NAJARRO, Mountain View, CA (US); Stephen WILLIAMS, San Carlos, CA (US); Kelly LI, Redwood City, CA (US); Chunfai LAI, Fremont, CA (US); Rui YANG, Fremont, CA (US); Larbi BEDRANI, South San Francisco, CA (US)

(21) Appl. No.: 19/145,454

(22) PCT Filed: Jan. 2, 2024
(86) PCT No.: PCT/US2024/010051

§ 371 Date: Jul. 2, 2025

Related U.S. Application Data
(60) Provisional application No. 63/436,655, filed on Jan. 2, 2023.

(51) Int. Cl. C12Q 1/70; C12Q 1/6806; C12Q 1/6825; C12Q 1/686

(52) U.S. Cl. CPC C12Q 1/701; C12Q 1/6806; C12Q 1/6825; C12Q 1/686; C12Q 2600/16

(57) ABSTRACT

Compositions for detecting the presence of monkeypox virus in a biological sample includes a monkeypox nucleic acid forward primer; a monkeypox nucleic acid reverse primer; and a monkeypox nucleic acid probe. Methods include a multiplex assay including a three-channel panel with a single assay per channel, that includes a monkeypox assay with a reporter and a quencher, a non-variola orthopox assay with a reporter and a quencher, and a RNase P assay with a reporter and a quencher. A kit for detecting monkeypox virus nucleic acid includes the assay components including the forward and reverser primers and probes for each of the three assays in the multiplex assay.

### FIELD

The present disclosure relates to compositions, methods, systems, and kits for specific detection, diagnosis, and differentiation of monkeypox virus from other orthopox viruses involved in infectious diseases. Differential detection of specific viral agents allows accurate diagnosis so that appropriate treatment and infection control measures can be provided in a timely manner.

### BACKGROUND

Monkeypox is a viral infection that is currently endemic to parts of central Africa, where there are two identified clades, endemic clade I and endemic clade II. A worldwide outbreak of clade II was reported to be first detected in London in May 2022 and by September 2022 more than 60,000 cases in more than 100 countries have been confirmed and the numbers continue to climb.

According to the World Health Organization (WHO), monkeypox is a viral zoonotic disease caused by monkeypox virus, a member of the orthopox virus genus of the Poxviridae family of viruses, and is transmitted by close contact with lesions, body fluids, respiratory droplets, and/or contaminated materials of an infected person or animal. The monkeypox virus is a deoxyribonucleic acid (DNA) virus most closely related to the smallpox virus. The virus is transmitted to humans as a result of close contact with infected animals, infected humans or contaminated inanimate objects.

According to the U.S. Centers for Disease Control and Prevention (CDC), monkeypox symptoms typically start within about three weeks of exposure and last about two to four weeks. Symptoms may include a rash on or near the genitals or other areas of the body that may look like pimples or blisters and may be painful or itchy. Other symptoms may include a fever, chills, swollen lymph nodes, exhaustion, muscle aches, headaches, and/or respiratory symptoms.

Given the global present and continuing emergence monkeypox virus, there is an urgent need to develop methods for the rapid detection and characterization of the virus so that appropriate infection control measures and treatment can be properly instituted in a timely manner. Monkeypox virus has a long incubation period, and it can take 4-21 days to develop symptoms after exposure. During that time, an infected person may be unknowingly spreading monkeypox to others. Moreover, it can be difficult to distinguish monkeypox based on clinical presentation since various conditions cause skin rashes. Epidemiologic factors are considered together with clinical presentation and ultimately testing to confirm disease. Laboratory confirmation of infection is accomplished using surface skin lesion samples that are analyzed employing nucleic acid amplification testing, such as real-time or conventional polymerase chain reaction (PCR) possibly followed by genetic sequencing. However, because the currently available tests for monkeypox require analysis of fluid from skin lesions, these tests are essentially confirmatory rather than diagnostic.

While detection products exist for detecting monkeypox viral infections, these suffer from the challenges with detection early in infection, differentiation between a monkeypox viral infection and infections with related non-variola orthopox viruses, as well as challenges relating to false positive and negative results. These challenges can contribute to the spread of infections, hinder accurate tracking of the disease, result in unnecessary delivery of or withholding of treatment and possibly hamper appropriate and timely treatment.

Antigen and antibody detection methods are not as reliable for the detection of a monkeypox viral infection due to the serological cross-reactivity of orthopox viruses.

Accordingly, there is an urgent need to develop detection products capable of overcoming some or all of the foregoing challenges and contribute to meeting the global need for robust and effective laboratory tools to timely diagnose and deliver treatment.

According to the instant disclosure, compositions, methods and kits address and overcome at least some of the foregoing problems in the art.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1A illustrates amplification plots for representative examples of monkeypox viral sequences according to the disclosure.

FIG. 1B illustrates PCR efficiencies for the amplification plots shown for the representative example of monkeypox viral sequences in FIG. 1A.

FIG. 2A illustrates amplification plots for representative examples of orthopox viral sequences according to the disclosure.

FIG. 2B illustrates PCR efficiencies for the amplification plots for the representative examples of orthopox viral sequences shown in FIG. 2A.

### DETAILED DESCRIPTION

All publications and patent applications cited herein are incorporated by reference in their entirety for all purposes to the same extent as if each individual publication or patent application were specifically and individually indicated to be so incorporated by reference. Although particular examples that reference specific target nucleic acids, formulations, and process steps are provided, it will be understood that these examples may be modified by using any of the formulations, components, and/or process steps described elsewhere herein, including by using any of the primers and/or probes described herein. Further, although the present invention has been described in some detail by way of illustration and example for purposes of clarity and understanding, it will be apparent that certain changes and modifications may be practiced within the scope of the spirit and substance of this disclosure and of the appended claims.

### Overview of Compositions, Systems, and Kits for Detection of Viral Sequences

Given the present and continuing spread of monkeypox virus, there is an urgent need to develop compositions, kits, methods, and the like for the accurate and rapid detection and characterization of monkeypox virus. In the case of monkeypox virus, for example, appropriate tracking can be implemented so that treatment and infection control measures can be properly instituted in a timely manner. Each late detected, misidentified or misdiagnosed instance of monkeypox virus infection further convolutes the epidemiological data and prevents the implementation of appropriate, informed solutions that may help control spread of the virus.

In some embodiments, the present disclosure relates to compositions, kits, and methods for detection of monkeypox virus. When an example “embodiment” or a particular “assay” is described herein, it will be understood that the features of the embodiment may be applicable to a composition (e.g., the particular physical components of an assay such as primers and/or probes, or a primer-probe set), a kit (e.g., primers and/or probes and additional buffers, reagents, etc.), or a method (e.g., a process for detecting target nucleic acids) as appropriate. For simplicity, many embodiments are presented by describing “assays”, but it will be understood that the associated methods of using the assays are also intended to form part of this disclosure.

In “multiplex” amplification embodiments, the formation of a plurality of separate and different amplification products or amplicons can be tracked over time by measuring a signal in one or more detection channels. The signal can be emitted by a detectable label, optionally a fluorescent label, attached to a primer and/or probe that selectively hybridizes to the amplification product or amplicon. In some embodiments, each channel is calibrated to preferentially or selectively detect a corresponding amplification product or amplicon, and the signal in each channel is used as a measure of concentration of the corresponding amplification product or amplicon. For example, in some embodiments, an amplification product or amplicon of the monkeypox DNA is detected in a first detection channel based on a first signal emitted by a first label attached to, or associated with, a first primer and/or first probe that selectively hybridizes to the amplification product or amplicon of the monkeypox DNA, and an amplification product of ribonuclease P is detected in a second detection channel based on a second signal emitted by a second label attached to, or associated with, a second primer and/or second probe that selectively hybridizes to the ribonuclease P DNA amplification product. Optionally, in “triplex” embodiments, an amplification product or amplicon of non-variola orthopox DNA is detected in a third channel based on a third signal emitted by a third label attached to a third primer and/or third probe that selectively hybridizes to the non-variola orthopox DNA amplification product or amplicon.

In some embodiments, the amplification product or amplicon of a positive control or reference sequence is detected in a fourth channel based on a fourth signal emitted by a fourth label attached to a fourth primer and/or fourth probe that selectively hybridizes to amplification product or amplicon of the positive control or reference sequence.

In some embodiments, a background noise is detected in a fifth channel based on a signal from a negative control. In a further embodiment, the negative control is water, for instance.

In some embodiments, a multiplex assay according to some embodiments includes a triplex assay. In some embodiments, the triplex assay includes a three-channel panels with each assay in a different channel. In some embodiments, the triplex assay includes assays for monkeypox (MPX) virus, non-variola orthopox (OPX) virus, and ribonuclease P (RNase P). In some embodiments, the triplex assay includes one forward primer, one reverse primer, and/or one probe for each of the MPX assay, the OPX assay, and the RNase P assay. In some embodiments, the primers and probes are selected from the target sequences shown in Tables 1, 2 and 3 below.

Several monkeypox virus panels are currently on the market for monkeypox polymerase chain reaction (PCR)-based tests. For example, Biosearch Technologies (Hoddesdon, UK) offers a monkeypox virus panel, a non-variola orthopox panel, and a human RNase P extraction control panel combination. However, the panels offered by Biosearch

Technologies only include individual primer and probes, and are not provided in the form of a reaction mixture as described in the context of this disclosure. One of skill in the art would appreciate the complexity in formulating a reaction mixture taking into account the interactions of the various components. RayBiotech, Inc. (Peachtree Corners, GA) offers a monkeypox virus real-time PCR kit. However, the kit offered by RayBiotech does not include an orthopox virus assay. GT Molecular (Fort Collins, CO) offers an All-in-One multiplex PCR test kit for human monkeypox virus.

As explained above, monkeypox virus may not be detected with the same efficacy or specificity using conventional diagnostic assays. This lack of resolution can prove problematic in attempts to track the spread and progression of monkeypox and/or requires more expensive and lengthy sequencing testing to identify the monkeypox virus specifically.

### Example Assays and Associated Components

Embodiments disclosed herein include primers and optionally probes useful for the detection of monkeypox virus and/or for distinguishing from non-variola orthopox viruses, in a sample (e.g., a biological or environmental sample). Such primers, oligonucleotides, and probes can be used in a nucleic acid assay (singleplex or multiplex) for detection and identification of one or more nucleic acid targets in a sample. The singleplex and multiplex assays described herein demonstrate a high level of sensitivity, specificity, and accuracy. In some embodiments, an assay is designed to detect and differentiate between monkeypox virus and other non-variola orthopox viruses. For example, an assay may be configured to detect the presence of monkeypox virus nucleic acid and nucleic acid of other non-variola orthopox viruses within a biological sample.

In some embodiments, an assay includes differentially labelled probes where at least one probe is configured to associate with, or configured to hybridize to, amplicons of a monkeypox sequence, and at least one, different probe is configured to hybridize to, or configured to associate with amplicons of a non-variola orthopox sequence. An additional labelled probe configured to hybridize to RNase P may be further included in the assay.

In some embodiments, assays are configured to detect an amplification product of a particular target region by detecting a signal from a label (i.e., a detectable label) or other signal-generating process, where the signal indicates formation of the amplification product. In some embodiments, the label is attached to, or otherwise associated with, the corresponding forward primer and/or reverse primer used to generate the amplification product. Additionally, or alternatively, the label is attached to, linked, or otherwise associated with, either directly or indirectly, a probe that hybridizes to or associate with a probe-binding sequence within the target region. In some embodiments, the target region is the region shown in Table 1. In some embodiments, the label is an optically detectable label. Alternatively, the label may be detectable non-optically, such as, for example, electronically, electrically, or by using nuclear magnetic resonance (NMR) spectroscopy, sound, radioactivity, and the like.

In some exemplary processes for detecting monkeypox virus, target DNA is first amplified by polymerase chain reaction (PCR). The reaction mixture includes a probe designed to target a sequence of monkeypox virus (MPX), a probe designed to target a sequence of non-variola orthopox virus (OPX), and a probe designed to target a sequence of RNase P. Each probe type is also associated with a different dye channel to enable differential detection. In an example embodiment, the MPX probe includes a FAM dye label, the OPX probe includes a VIC dye label, and the RNase P probe includes a JUN dye label. The probes may be configured as TaqMan probes, which are known in the art and described in greater detail below. When the probe is able to hybridize to a target downstream from a primer, the exonuclease activity of the polymerase during subsequent primer extension separates the dye label from the quencher to increase the dye signal.

Disclosed herein are primers and probes that hybridize to monkeypox virus, as well as primers and probes that hybridize to non-variola orthopox virus and RNase P, that may be utilized in an assay that can beneficially detect monkeypox virus.

In some embodiments, the assay is a multiplex assay including at least a first set of primers and probes. In some embodiments, the first set of primers and probes include monkeypox primers and probes. In some embodiment, the monkeypox primers and probes include one or more sets of those shown in Table 1. Table 1 lists exemplary MPX forward primers (corresponding to SEQ ID NO:16-SEQ ID NO:70) and MPX reverse primers (corresponding to SEQ ID NO:71-SEQ ID NO:125), and MPX probes (corresponding to SEQ ID NO: 126-SEQ ID NO:180) that may be utilized in conjunction with the corresponding MPX forward and reverse primers. For example, in some embodiments, an assay can include a forward primer and reverse primer in a particular “Set” row of Table 1 and a probe in the same “Set” row of Table. In other embodiments, an assay can include one or more MPX forward primers selected from SEQ ID NO: 16-SEQ ID NO:70, one or more reverse primers selected from SEQ ID NO:71-SEQ ID NO:125, and one or more probes selected from SEQ ID NO:126-SEQ ID NO:180.

| | SEQ | | SEQ | | SEQ | |
|---|---|---|---|---|---|---|
| | ID | | ID | | ID | |
| Set | NO: | Forward Primer | NO: | Reverse Primer | NO: | Probe |
| 1 | 16 | ATGTGGGATTTGGTAGACCTCCTA | 71 | TGAATAGCCCCAGACTGTATGCT | 126 | TTGTTAGCGGGTATCCC |
| 2 | 17 | GCGCTCGATGGGACATTTC | 72 | ACACTGCAGTTAGTTTTACCACCAT | 127 | AACGTAGACCGTTTAATAAT |
| 3 | 18 | GCAAGCAGCCAGTATAGAAATTGTT | 73 | GTGGTACAGCACGTAGTGACTATT | 128 | CTCCCATGGAATGACC |
| 4 | 19 | ACAGAAGCAGCTGCAGCAA | 74 | CGGATGATCTGCACAGAACTCA | 129 | CTGTTGATGCACAGTCTGC |
| 5 | 20 | TCGGTTTTGGTGAAATCCAAGGAT | 75 | CAGCGCATCTATCTAACTACATCGT | 130 | CAATCCACAGACAAAACA |
| 6 | 21 | CGTAAGAACACGATGGAGAGTAATCG | 76 | CGTGTTGGCTGCAAAATATATCCA | 131 | CCGGGATCTGCGATTAA |
| 7 | 22 | CGAGTTCCCATAGCTGGTTCAAAAT | 77 | TGCGACGTTGTATACGCCATAA | 132 | ATGTAACTTGAGAAAATCC |
| 8 | 23 | CGATGGCTCTTGAAACCAAGGT | 78 | CTAATGAAAAAGACTTCTCACCGATCGA | 133 | TTCCAACCGGACTCATTG |
| 9 | 24 | GGCATCTTGAATAGAAACAACACCAT | 79 | CCGACTGGTTATCCGGAATCTG | 134 | CCGAGATCGTAGAGGTTC |
| 10 | 25 | CGTTCGATTAACCCAACTCATCCAT | 80 | GTGCAACCGATGATACTTCACTCA | 135 | ATGTGTCTGAATCGATAACTC |
| 11 | 26 | CGGGAGAGTCCTTACCTTGTC | 81 | GAATCCTCTGATGGAAACACTGTGA | 136 | CACGCTGGACAATCTA |
| 12 | 27 | TCATAGGTCTAGCCACAGAAACCA | 82 | CTGGATGCTCTCAAGTTATGACAGA | 137 | ACGTAGGTCTAAATCCAACTAT |
| 13 | 28 | GTTCGGTGCAGCTAGTACTCTTAA | 83 | GACTTTTTATGAAGAGCCGCGTTTA | 138 | ACGTTCGGAGATAATAAAGCA |
| 14 | 29 | CTCACCTGTCATGTCTGGTAATTCA | 84 | GCAATCCGAATGTGTCCATCCA | 139 | CTGTTGAAGCATTTCTTG |
| 15 | 30 | CGTTAGTAAAACATGGCGAGGAAAT | 85 | TCCGCCGATGTGAACGT | 140 | CTTGACTTTTTCACAAACTC |
| 16 | 31 | GATCCTCCTTGTATTTGTCACATTCCT | 86 | GTCGTAATGTCGAATGGGAACTGT | 141 | CCAGACTTCGTATGGATATC |
| 17 | 32 | CCTGCTCGGTTACTTCTGTGTA | 87 | GCATGCTTCCCATATTCTACATAGGT | 142 | ATACACTGTGTCAAAACTG |
| 18 | 33 | ACCACAGACTGAAAGTATCTCTACCT | 88 | GGCTATTATCTTCTTTACGTAGTGATCGT | 143 | AAGATGTCGGAGTTCTC |
| 19 | 34 | GCATTAGTAAGTGCTGCAAAACAGA | 89 | GGTGCATATTGCAAACTGGTCATTA | 144 | CAAAGTCGCAGTATTGTTAAC |
| 20 | 35 | GCCGTGGGATAATGTTAAGGATTGT | 90 | GGCCGAAATCTCTCTGATGGTTTT | 145 | AACAACGGATGATCTCTG |
| 21 | 36 | AGGCGTTTATCATATCTGTGATTTCGT | 91 | ACGTGTACCGTATCCATAGGTGAT | 146 | ACTGGGTTGTAATGTTATCC |
| 22 | 37 | GGGTGAAGTTATTCCCCTCATCA | 92 | CCATAGCATACGTGTTCTCATCCAT | 147 | TTGGTCAGAAATTTTG |
| 23 | 38 | ACTCCCTGAATACCGTGACTGA | 93 | CGCTTTCTCTATGGGTCCATCTAT | 148 | ATCCTGTGGGTTTCTC |
| 24 | 39 | CGTATAATGCCGTATCCCCAAAAGT | 94 | GCTCCAAACATATGGATGCGAGATA | 149 | AATAATCAGGACGAATAATTC |
| 25 | 40 | GCGATGACAGATCCCCTATCC | 95 | GCAAATGTCAACGCAGTAATGAGT | 150 | CACGTCAAGACATGTATATC |
| 26 | 41 | CTATTGTGTCACCTCTCACCAGAAA | 96 | GACCATCATTTGTAGGGCGTCTAG | 151 | TCGTCGTCTAGTAGCTCCT |
| 27 | 42 | GCATTGATGACACTGGATGATTTGG | 97 | CCCGAATCGGAGTCTACTTTAAGTT | 152 | ACAGTATGGAGACATTGATCT |
| 28 | 43 | GGGTAAACTGTCTCCAATTTTTGTGT | 98 | CACGCATACCTTTTCAATGAGCATT | 153 | CAGTGGCACTAATCC |
| 29 | 44 | CGGAATCGCAAACCACTTGTG | 99 | ACGGGAATGTACTATCTACGTACGAA | 154 | CCGCTCCCATTCAATTCACAT |
| 30 | 45 | GAAGAGTATACAGAAGCAGCTGCA | 100 | CGGATGATCTGCACAGAACTCA | 155 | CCACTAGTACAGAAGTTGC |
| 31 | 46 | TGAATGATATGGCCGCTCTAGGA | 101 | GCCTTTCCCATTTAACCACATAGTCT | 156 | TTCAATAGGAGGACACTTTG |
| 32 | 47 | GGACAAATCTGGAAAAACAACACAATGT | 102 | GTGGATCGCTGAGGAAAGTTAAGAT | 157 | CCGGCAAACACGATAAA |
| 33 | 48 | GGTCCTCCTTATCTCCTCCAGTAG | 103 | CCGCGTACGACAAGCATTTG | 158 | CATGTGGTTCTTCAATACC |
| 34 | 49 | GCTACTTGGTGGGATACTGAAGGA | 108 | CGATTAGTGGCGTGACACCAT | 159 | TCGGGTTCGACATCCAC |
| 35 | 50 | CGGTACACGTACCAGCATTTGTA | 105 | CGAGAGTGGAGATATTACAGGAGAAGA | 160 | TTCTGCGCAATGCCA |
| 36 | 51 | CCAATGCGTCCATGTTGTCTATTT | 106 | ACTGGTGGTAGAACTGTTCCTAGAA | 161 | CAACAGGTCGCTATATCC |
| 37 | 52 | CGAGCGTGAATAACTTACAGATGGA | 107 | TTTTGCTCTATTGACGGGAACAGT | 162 | AAGCAATAGTGATAGATTTTC |
| 38 | 53 | CCGCAATACTCCTCATTACGTTTCA | 108 | TGCGCTTCTCAATTGGATAATCTATGT | 163 | AACAAAATGGGCATTCTAG |
| 39 | 54 | GCAGAAATAACCGAATTAGTCGTCGAA | 109 | CTCGTCCTCAATGAATGTCTCCAT | 164 | CCCCATTCCTTTACAATGAC |
| 40 | 55 | GGTTCGGATACCTTTACATCTCACA | 110 | CGATCGCGTCTCTACCTGATTACTA | 165 | ACTTAGACAAGCCTGTAAATG |
| 41 | 56 | CGTGCGATTCAGAACACACTTTTC | 111 | GTTTGACTTTATCGTACGCGTCATT | 166 | CCTCTCTACCAGAAGGTTG |
| 42 | 57 | CAACAGGTCGCTATATCCACCAATA | 112 | CCCGAAAATGAATTGCGTGACTAT | 167 | CAGTTCTACCACCAGTAATT |
| 43 | 58 | GTCCTTCCACCAGCATCTAGTTG | 113 | GATTCCTTGTAGATGAACCCACAGT | 168 | AAGCTTTGTATCCCATATATTG |
| 44 | 59 | TCGGATTTTGTGGGTTCTTGATCTT | 114 | TGTTGATCGGTATTTGTGTAGCAGTT | 169 | TCGCCATCCTATACACGCTG |
| 45 | 60 | GTGTTTCGAATGCCTCGTACAAG | 115 | CGCAATTCTAAATCGTTGGTCAAAGA | 170 | CTATGGCATCCTTGAAATC |
| 46 | 61 | CATATTCTCACACCGTCTCTTCCA | 116 | GTGTCGTTGACTGGATACAGGTTAA | 171 | TACGGGTTCGCATTTAT |
| 47 | 62 | GGCATAATCCGGATGTTGTGTAGTA | 117 | GGAGTAACAACGGTAGAATTGGACA | 172 | TCTGGCAGCCGAAATA |
| 48 | 63 | AGAAAGTTACGTTTACATGTGATTCAGGAT | 118 | GCACATTTTCTTGCATGGATTTTCG | 173 | TCGCAGACAGCATTTG |
| 49 | 64 | GTTTGCTGCTAATCGCGACA | 119 | CCCGGGAGACACGGATTTATTAA | 174 | ACGTAACGTCAASACTTT |
| 50 | 65 | CTCAACAAGTTCTCAATACCCCAATAGA | 120 | GTGGATATAGCGACCTGTTGGAAAT | 175 | CACCCAATGCGTCCATG |
| 51 | 66 | GCTGGCGACCATCATATTATCTCT | 121 | GATGAAGCGATTGAAATAGGACTAAAGTC | 176 | TTCTCGCAACTGTCTTTG |
| 52 | 67 | CAAGATCATTTCTACGTCCTATGGATGTG | 122 | GTAGCCAACTCAGTATATGGTCTGATG | 177 | ACGCTTCGGCTAAGAGT |
| 53 | 68 | GACACGCTGGACAATCTAGTATTCA | 123 | TGGATTCACCAAATGCCCAAAGAT | 178 | CCTCTGATGGAAACACTG |
| 54 | 69 | CGACGGTACAACATTCCAAAGATC | 124 | AGCTGAGAAGGAAGGACCATATGA | 179 | ACGCCAGTAACGGTATTC |
| 55 | 70 | TGTGGTTAAATGGGAAAGGCTAGAAAA | 125 | CCATAAATCACCATGTTTGACACGTT | 180 | CTGTCGCCGTCTATTC |

In some embodiments, the multiplex assay further includes a second set of primers and probes. In some embodiments, the second set of primers and probes include non-variola orthopox (OPX) primers and probes. In some embodiments, the OPX primers and probes are known OPX primers and probes. In some embodiments, the OPX primers and probes include the sequences shown in Table 2, which were designed by the Centers for Disease Control & Prevention (CDC) Poxvirus & Rabies Branch (PRB) (Test Procedure: Non-variola Orthopoxvirus Generic Real-time PCR Test, Rev. No. 2, Issued Jun. 6, 2022).

TABLE 2
OPX Primers and Probes

| SET 1 | SEQ ID NO: | Sequence |
|---|---|---|
| Forward | 4 | GAGTCGGAGCCATCTGGTTTAATAT |
| Primer | | |
| Reverse | 5 | CCACCTTTCACTCAACACCTTCTTA |
| Primer | | |
| Probe | 6 | AACCACCGATAAATCTAG |
| SET 2 | SEQ ID NO: | Sequence |
| Forward | 7 | AGTTCCATTAGCCTTTCCACTTCTG |
| Primer | | |
| Reverse | 8 | CCAATGATCATCGGTGAGCCTATTA |
| Primer | | |
| Probe | 9 | CTGGTAAAGAACCAATTTCT |
| SET 3 | SEQ ID NO: | Sequence |
| Forward | 10 | TCAACTGAAAAGGCCATCTATGA |
| Primer | | |
| Reverse | 11 | GAGTATAGAGCACTATTTCTAAATCCCA |
| Primer | | |
| Probe | 12 | CCATGCAATATACGTACAAGATAGTAGC |
| | | CAAC |

In some embodiments, the multiplex assay further includes a third set of primers and probes. In some embodiments, the third set of primers and probes include RNase P primers and probes. In some embodiments, the RNase P primers and probes include the sequences shown in Table 3.

TABLE 3
RNase P Primers and Probe

| | SEQ ID NO: | Sequence |
|---|---|---|
| Forward Primer | 13 | AGATTTGGACCTGCGAGCG |
| Reverse Primer | 14 | GAGCGGCTGTCTCCACAAGT |
| Probe | 15 | TTCTGACCTGAAGGCTCTGCGCG |

In some embodiments, multiple assays each corresponding to a different organism can be combined to create an assay panel designed to specifically detect monkeypox virus.

In embodiments, each probe includes a detectable label (e.g., a fluorescent reporter molecule) and a quencher molecule capable of quenching the fluorescence of the reporter molecule. Typically, the detectable label and quencher molecule are part of a single probe.

In one embodiment, the OPX probe is labelled with VIC, the monkeypox probe is labelled with FAM, and the RNaseP probe is labelled with JUN. However, these labels may be swapped, or other suitable labels, as known in the art and/or as described elsewhere herein, may be additionally or alternatively be utilized for a reference probe or a mutant/variant probe, including, but not limited to, ABY, Alexa Fluor dye labels (e.g., AF647 and AF676), as well as the dyes discussed below.

In some embodiments, the multiplex assay further includes a positive control. In an embodiment, the positive control includes templates specific to monkeypox virus, non-variola orthopox virus targets, and human RNase P regions targeted by the assay. In some embodiments, the positive control includes the sequences shown in Table 4.

TABLE 4
Positive Control

| | SEQ | |
|---|---|---|
| | ID | |
| Assay | NO: | Sequence |
| MPX | 1 | TTCAGGTTTTGGTGGAAGTGTAACTGCTACTTG |
| | | GTGGGATACTGAAGGATATTTCAGAGAGTTGTG |
| | | GATGTTCGGGTTCGACATCCACCGATGGTGTCA |
| | | CGCCACTAATCGGTTCGGTAACGTCTGTGGATG |
| | | GAGG |
| OPX | 2 | TGTGCAACTCTTAGCCGAAGCGTATGAGTATAG |
| | | AGCACTATTTCTAAATCCCATCAGACCATATAC |
| | | TGAGTTGGCTACTATCTTGTACGTATATTGCAT |
| | | GGAATCATAGATGGCCTTTTCAGTTGAACTGGT |
| | | AGCCTGTTTTAACATCTTT |
| RNase P | 3 | GGGACTTCAGCATGGCGGTGTTTGCAGATTTGG |
| | | ACCTGCGAGCGGGTTCTGACCTGAAGGCTCTGC |
| | | GCGGACTTGTGGAGACAGCCGCTCACCTTGGCT |
| | | ATTCAGTTGTTGCTAT |

### Sample Collection

The disclosed compositions, kits, and methods are configured to detect viral nucleic acid from a sample. According to the instant disclosure, the disclosed compositions, kits, and methods are configured specifically for detection of monkeypox virus from a sample that includes material extracted from lesion material wherein DNA from the virus may be present. Accordingly, a sample can be extracted from acceptable specimen types including, but not limited to, lesion fluid on a dry swab, lesion fluid swab in viral transport media, lesion fluid on a slide, lesion crust, or lesion roof. In some embodiments, the sample is a human sample. In some embodiments, the sample is a non-human sample. For instance, the sample may be from a non-human species such as a rodent (e.g., prairie dogs, squirrels, chinchillas), a carnivore (e.g., dogs, cats), an insectivore (e.g., hedgehogs, shrews), a non-human primate (e.g., monkeys, apes), etc. In most instances, the monkeypox virus is detected by analysis of swabs or fluid obtained from swabs, such as lesion swabs. In some alternate embodiments, it should be appreciated that the monkeypox virus may also be detected by analysis of other swabs, such as, for example, throat swabs, nasal swabs, nasopharyngeal swabs, cheek swabs, saliva swabs, or other swabs, or urine samples, saliva samples, or other clinical samples. Such other samples may be collected with a collection device such as a tube, a dish, a bag, a plate, or any other appropriate container.

The sample can be collected by a healthcare professional in a healthcare setting, but in some instances, the sample is collected by the patient themselves or by an individual assisting the patient in self-collection. For example, a lesion swab has heretofore served as a standard for obtaining a patient sample to be used in clinical diagnostics for a potential pox virus. Such swabs are often used by a healthcare professional in a healthcare setting. Other samples can similarly be obtained in a healthcare setting with the assistance or oversight of a healthcare professional. However, in some instances, self-collection of a sample can be more efficient and can be done outside of a healthcare setting.

In some embodiments, the sample is a raw lesion sample collected-whether by self-collection or assisted/supervised collection-by contacting a sterile swab to a patient lesion. The lesion sample may be extracted from the swab into viral transport media (VTM) or universal transport media (UTM) or tested directly from the swab. In some embodiments, the tip of the swab holding the lesion sample can be placed into a sealable container containing VTM or UTM to extract a sufficient portion of the lesion sample from the swab before removal of the swab and closing/sealing the container. In other embodiments, the swab holding the lesion sample can be collected directly into a sealable container without any preservation solution or other fluid or substance in the container prior to receipt of the lesion sample within the container or because of closing/sealing the container. The swab storage tube/device or extracted sample collection tube/device may be a component of a self-collection kit having instructions for use, such as sample collection instructions, sample preparation or storage instructions, and/or shipping instructions.

In other embodiments, the sample is collected-whether by self-collection or assisted/supervised collection-in a sterile tube or specifically-designed collection device. The collection tube/device may be a component of a self-collection kit having instructions for use, such as sample collection instructions, sample preparation or storage instructions, and/or shipping instructions. The sample can be collected directly into a sealable container without any preservation solution or other fluid or substance in the container prior to receipt of the sample within the container or because of closing/sealing the container.

In some embodiments, the sample is pre-treated prior to use. This can include, for example, heating the sample, such as by placing the raw sample on a heat block/water bath set to a temperature of 95° C. for 30 minutes, followed by combining the heat-treated sample with a buffer or lysis solution. The buffer or lysis solution can include, for example, any nucleic-acid-amenable buffer such as tris-borate-ethylenediaminetetraacetic acid (TBE) and may further include a detergent and/or emulsifier such as the polysorbate-type nonionic surfactant Tween-20.

In some embodiments, a nucleic acid fraction of the sample (e.g., obtained by a swab) can be extracted and used for downstream analysis, such as qPCR. In some embodiments, the sample is a raw lesion sample. As provided above, the raw lesion sample can be self-collected (e.g., within a sterile swab collection device) or collected from the patient by any other individual in proximity to the patient. In some embodiments, the swab of the raw lesion sample is collected directly into a sealable container without any preservation solution or other fluid or substance in the container prior to receipt of the lesion sample or because of closing/sealing the container. The disclosed embodiments for detecting viral nucleic acid from a sample can be adapted to detect viral nucleic acid directly from the lesion sample, or in alternative embodiments, the sample can undergo a specific DNA purification and/or extraction step prior to its use in a detection assay (e.g., qPCR). Thus, it should be appreciated that in some embodiments, a patient sample (e.g., a lesion swab) can directly serve as sample input for subsequent downstream analyses, such as PCR, and this can be accomplished, in some embodiments, with no nucleic acid purification and/or extraction step prior to its use. In some embodiments, the sample used in subsequent downstream analyses is a heat-treated lesion sample as described herein.

In some implementations, viral nucleic acid may be detected directly from a raw or crude sample. For example, a raw lesion sample can be collected from the patient and heat-treated, such as by placing the raw lesion sample on a heat block/water bath set to a temperature of about 95° C. for 30 minutes. The heating step can provide many benefits, including, for example, denaturing nucleases such as RNase within the lesion sample that may interfere with accurate assessments of viral presence. Heating a raw sample can also make the sample more amenable to manipulation with laboratory equipment such as pipettes. The high heat can also cause thermal disruption of any prokaryotic and eukaryotic cells present in the lesion sample and can also disrupt enveloped viruses and/or viral capsids present in the lesion sample and thereby increase accessibility to any viral nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of viral nucleic acid within the sample via nucleic acid amplification reactions (e.g., PCR, qPCR, or the like). The lysis solution can include a nucleic-acid-amenable buffer such as TBE (and/or suitable alternative known in the art) combined with a detergent and/or emulsifier such as Tween-20, the polysorbate-type nonionic surfactant (and/or suitable alternative known in the art). The detergent and/or emulsifier can promote better mixing of the reagents and may also act to increase accessibility to any viral nucleic acid within the sample (e.g., by removing lipid envelopes from virions).

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the sample/buffer mixture. In other embodiments, the sample is directly combined with a buffer/detergent mixture.

As a nonlimiting example, a set of patient samples can be prepared as compositions for downstream analysis and detection of a viral sequence by adding a volume of heat-treated sample for each patient into one (or a plurality) of wells in a multi-well plate. A volume of a buffer/detergent mixture (e.g., TBE+Tween-20) can then be added to each well containing a patient sample. Alternatively, a multi-well plate can be loaded with a volume of a buffer/detergent mixture into which a volume of heat-treated sample is added. Once combined, this probative template solution can be used immediately or stored for later analysis. Such probative template solutions can also be combined with PCR reagents (e.g., buffers, deoxynucleotide triphosphates (dNTPs), master mixes, etc.) prior to or after storage.

In some embodiments, a sample is obtained from multiple organisms (e.g., a plurality of individuals or patients) and the multiples samples are pooled together to make a single pooled sample for testing. In some embodiments, a sample may be obtained from at least two different organisms or individuals for pooling together to form a single sample for testing. In some embodiments, a sample may be obtained from between 2 to 10 different organisms or individuals for pooling together to form a single sample for testing. In some embodiments, a sample may be obtained from 2, 3, 4, 5, 6, 7, 8, 9, or 10 different organisms or individuals for pooling together to form a single sample for testing. In some embodiments, a sample may be obtained from up to and including six different organisms or individuals for pooling together to form a single sample for testing. For example, a sample used for testing, according to the methods and compositions described herein, may comprise a multiplicity of samples obtained from different organisms or individuals (e.g., 2, 3, 4, 5, or 6 different individuals) which are combined together to form a single “pooled” sample used for subsequent detection of a pathogen such as monkeypox virus.

### Nucleic Acid Amplification and Detection

Amplified or amplification products (“amplicons”) resulting from use of one or more embodiments described herein can be generated, detected, and/or analyzed using any suitable method and on any suitable platform. In some embodiments, monkeypox virus or other target organism is detected by analysis of swabs, or fluid obtained from swabs, such as lesion fluid on a dry swab, lesion fluid swab in viral transport media, lesion fluid on a slide, lesion crust, or lesion roof. Monkeypox virus or other target organisms may additionally or alternatively be detected by analysis of saliva samples, buccal samples, nasal samples, nasal pharyngeal samples, blood samples, urine samples, semen samples, or other biological samples.

In some embodiments, the nucleic acid assays as described herein can be used to detect, identify, characterize, quantify, or otherwise measure one or more nucleic acid targets in a sample. In some embodiments, the nucleic acid targets may be single-stranded, double-stranded, or any other nucleic acid molecule of any size or conformation. Optionally, the nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods, including methods discussed below for detecting nucleic acid targets in a sample. In some embodiments, the PCR assays can be real time PCR or quantitative (qPCR) assays. In some other embodiments, the PCR assays can be end point PCR assays. Nucleic acid markers may be detected by any suitable means, including means that include nucleic acid amplification (e.g., thermal cycling amplification methods including PCR, and other nucleic acid amplification methods; isothermal amplification methods, including LAMP, etc.) and any other method that can be used to detect the presence of nucleic acid markers indicative of a disease-causing organism in a sample.

In some embodiments, the primers described herein are used in nucleic acid assays at a concentration in the range of about 100 nM to 1 mM (e.g., 300 nM, 400 nM, 500 nM, etc.), including all concentration amounts and ranges in between. In some embodiments, the probes described herein are used in a nucleic acid assay at a concentration in the range of about 50 nM to 500 nM (e.g., 75 nM, 125 nM, 250 nM, etc.), including all concentration amounts and ranges in between.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels may include, but are not limited to, 6FAM, ABY, VIC, JUN, FAM. Suitable quenchers may include, but are not limited to, MGB, QSY (e.g., QSY7 and QSY21), BHQ (Black Hole Quencher), and DFQ (Dark Fluorescent Quencher).

In some embodiments, control sequence primers and/or probes (e.g., VIC-labeled probes), such as for amplification and/or detection of non-variola orthopox virus control sequences, are included in the multiplex assays using primer/probe sequences disclosed herein (and may be included as singleplex assays as well).

In some embodiments, control sequence primers and/or probes (e.g., JUN-labeled probes), such as for amplification and/or detection of bacteriophage MS2 or human RNase P control sequences, are included in the multiplex assays using primer/probe sequences disclosed herein (and may be included as singleplex assays as well).

In some embodiments, array-formatted assays can be run as a singleplex assay or as multiplex assays. In some embodiments, a panel of different assays may be formatted onto an array or a multi-well plate. In some embodiments, the panel can include one or more assays containing at least one primer-set and a probe of Table. 1 present in at least one well of an array or a well of a multi-well plate. In some embodiments, the panel includes assays for non-variola orthopox virus and RNase P. In some embodiments, the disclosed methods include using the panel to profile microorganisms present in a sample taken from an organism (e.g., human) and determining the profile of microbiota present in the organism's sample. Optionally, the disclosed methods can include diagnosing an infection present in an organism (e.g., human) from which a sample is taken.

In some embodiments, the panel of qPCR assays can be used simultaneously to test a single patient sample or a single pooled sample comprising multiple patient samples, with each assay run in parallel in array format (“array formatted”). Optionally, different qPCR assays specific for each of the following target assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use.

In some embodiments, the panel of qPCR assays includes at least one qPCR assay for detecting monkeypox virus. In some other embodiments, the panel of qPCR assays includes at least one qPCR assay for detecting monkeypox virus, in addition to, at least one qPCR assay for detecting a non-variola orthopox virus. Each qPCR assay can include a forward primer and a reverse primer for each target, and a probe.

In some embodiments, the multiplex assay includes, at least, a forward primer and a reverse primer that hybridizes to a target gene sequence within the monkeypox genome, a forward primer and a reverse primer that hybridizes to a target gene sequence within the non-variola orthopox genome, as well as, a forward primer and a reverse primer that hybridizes to a gene coding for a RNA subunit of RNase P.

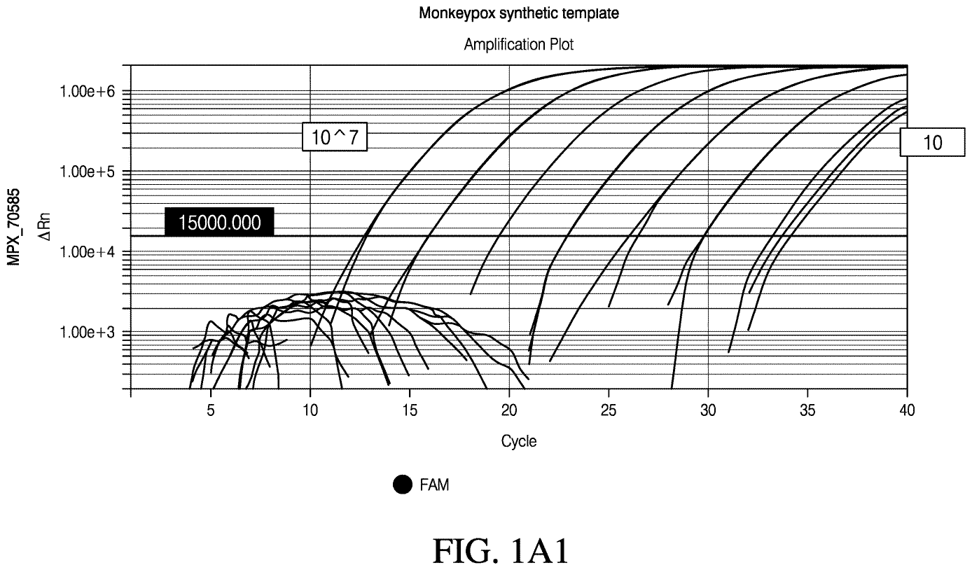

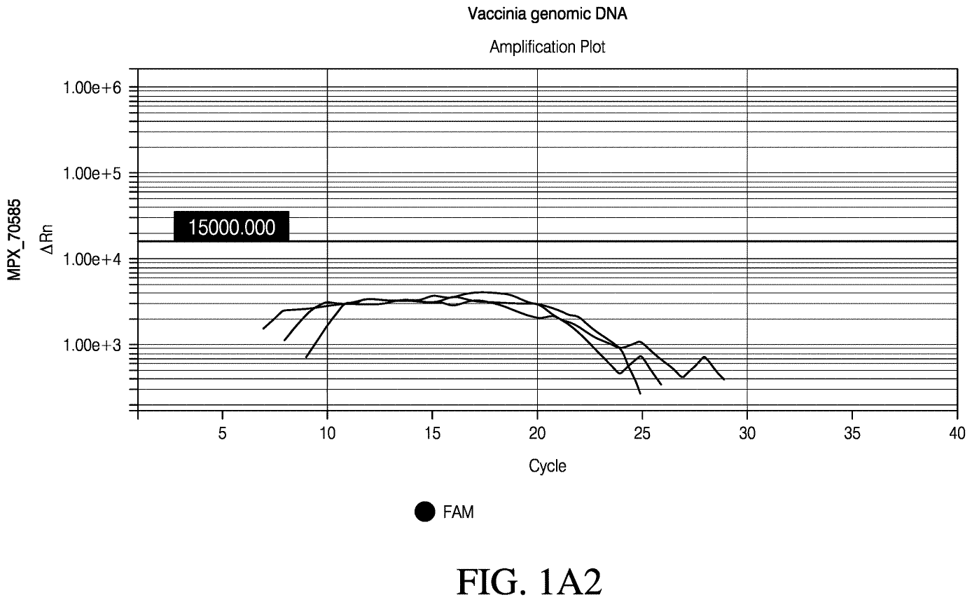

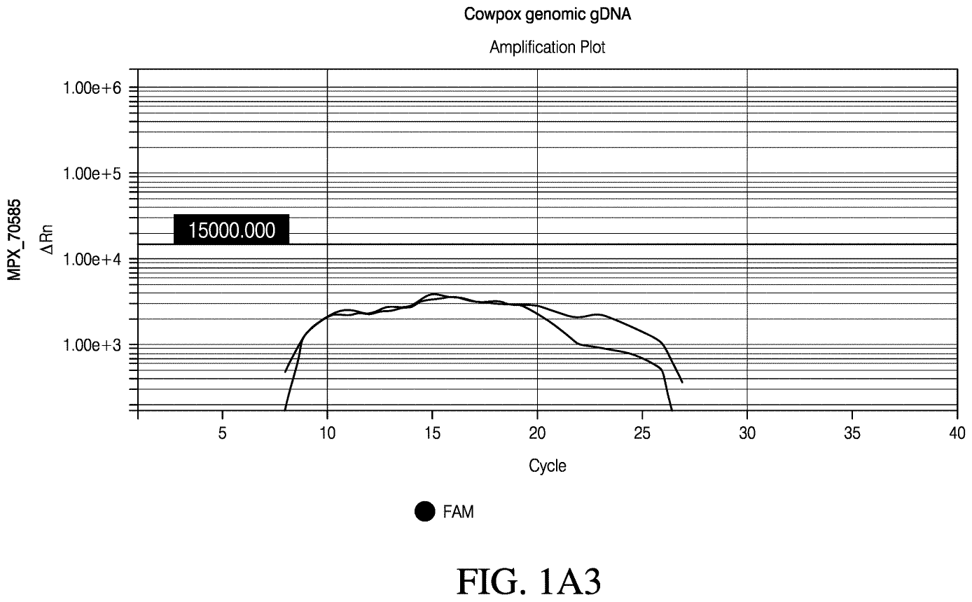

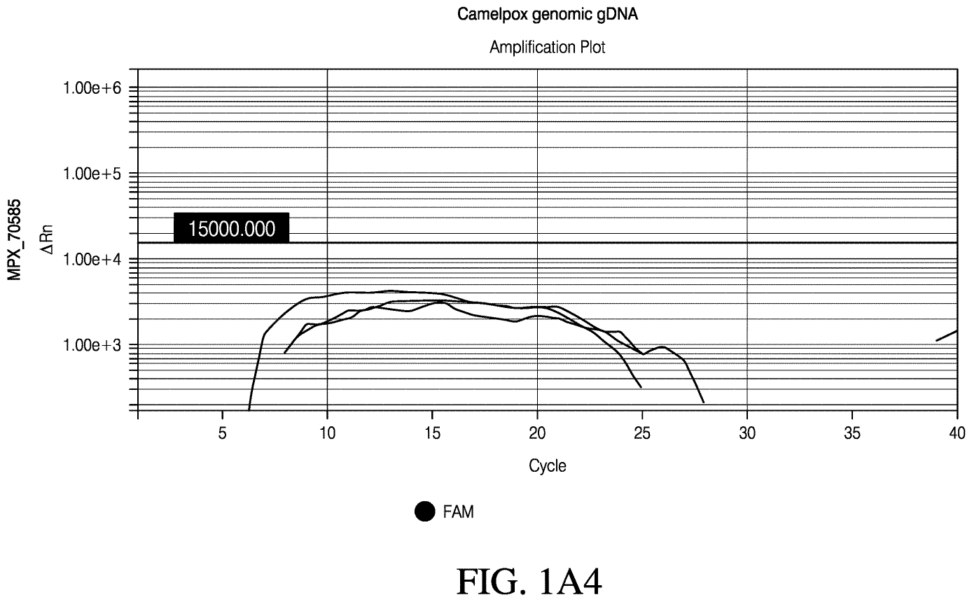

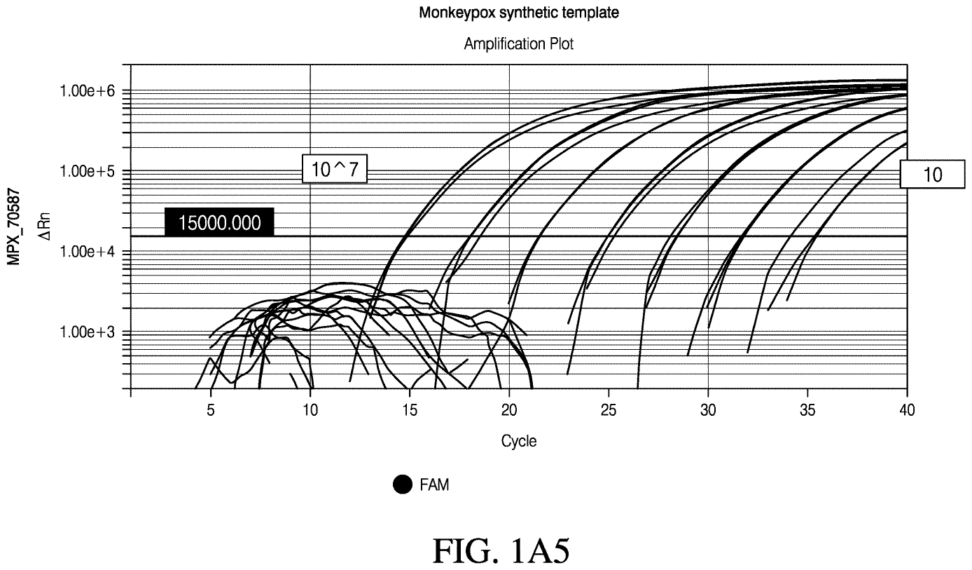

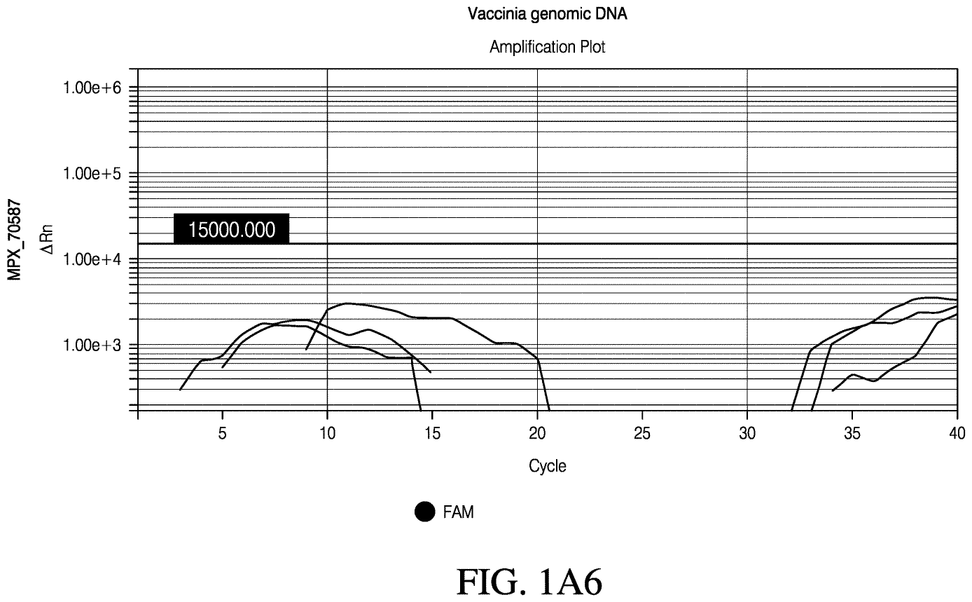

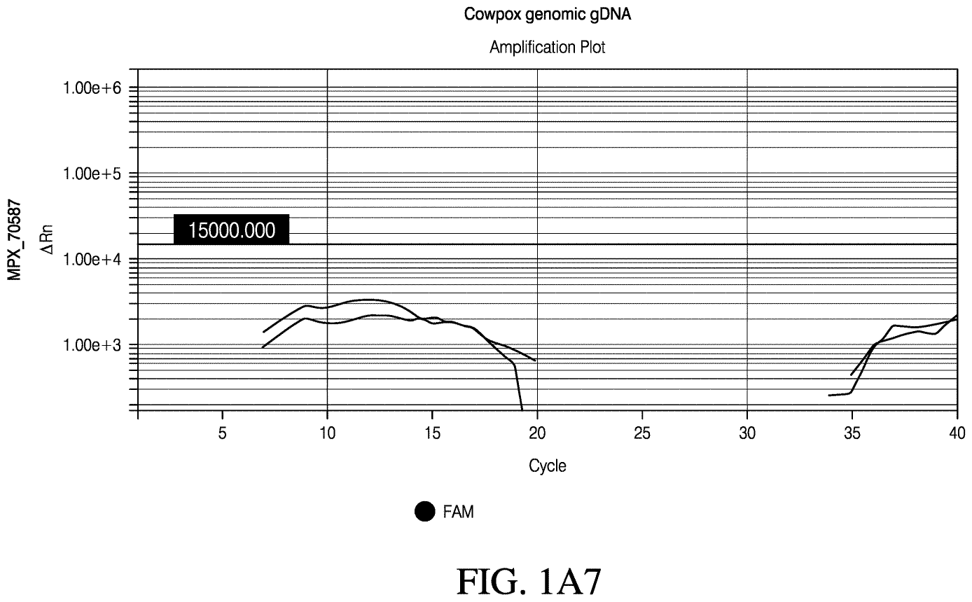

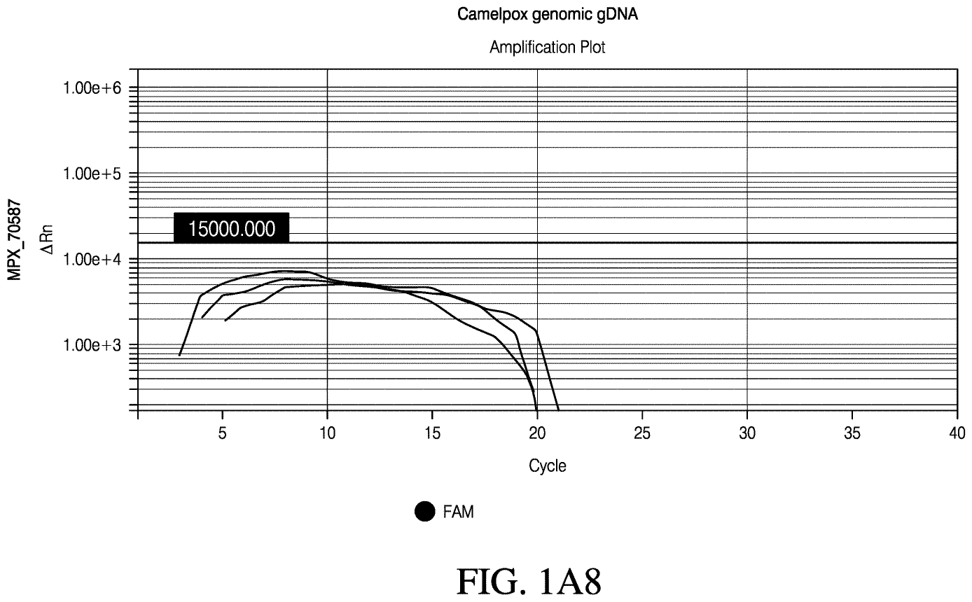

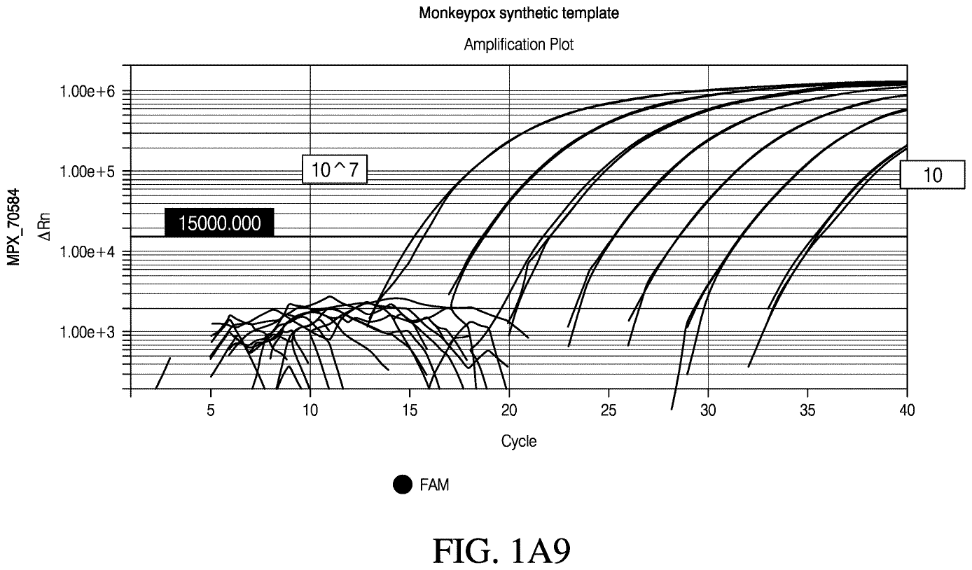

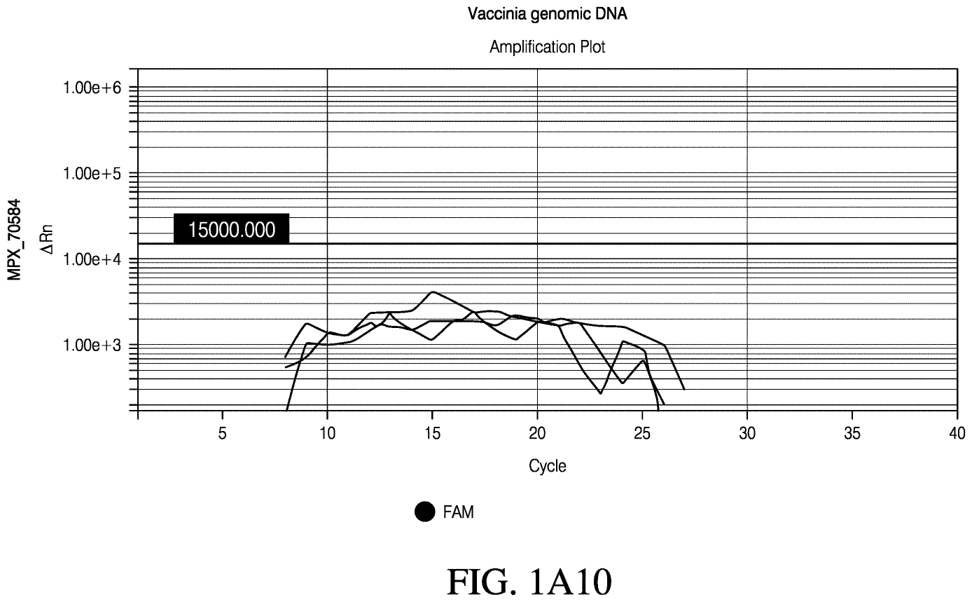

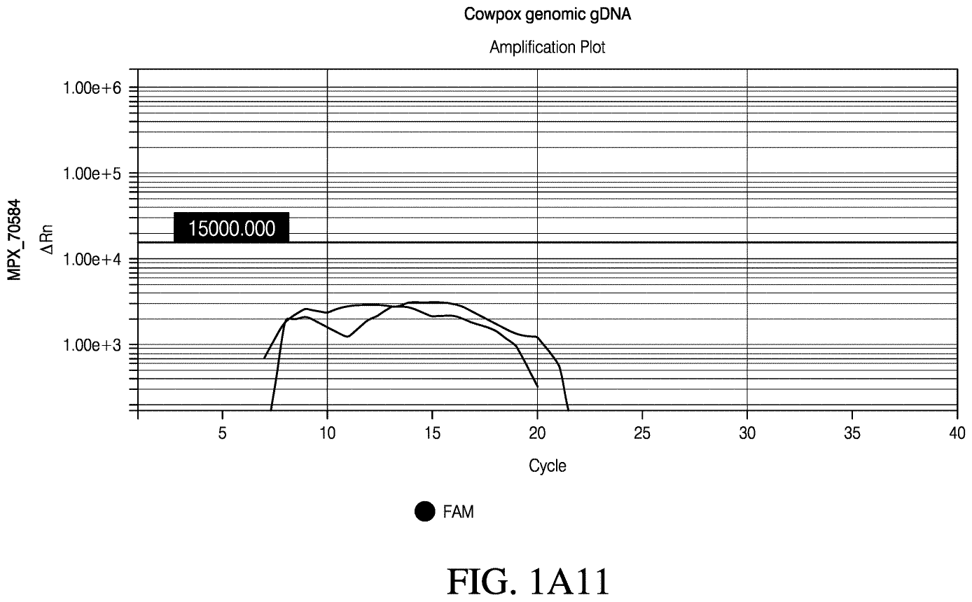

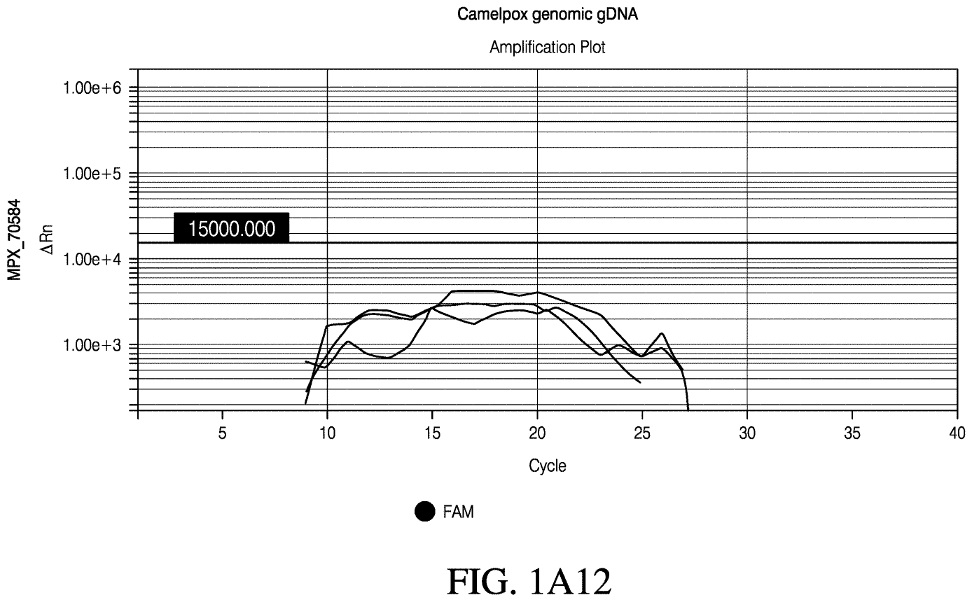

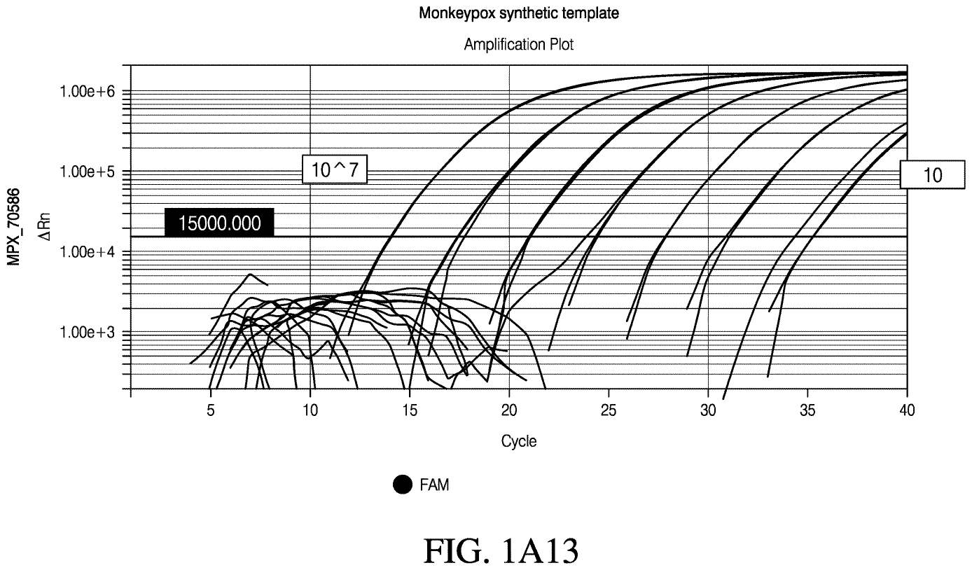

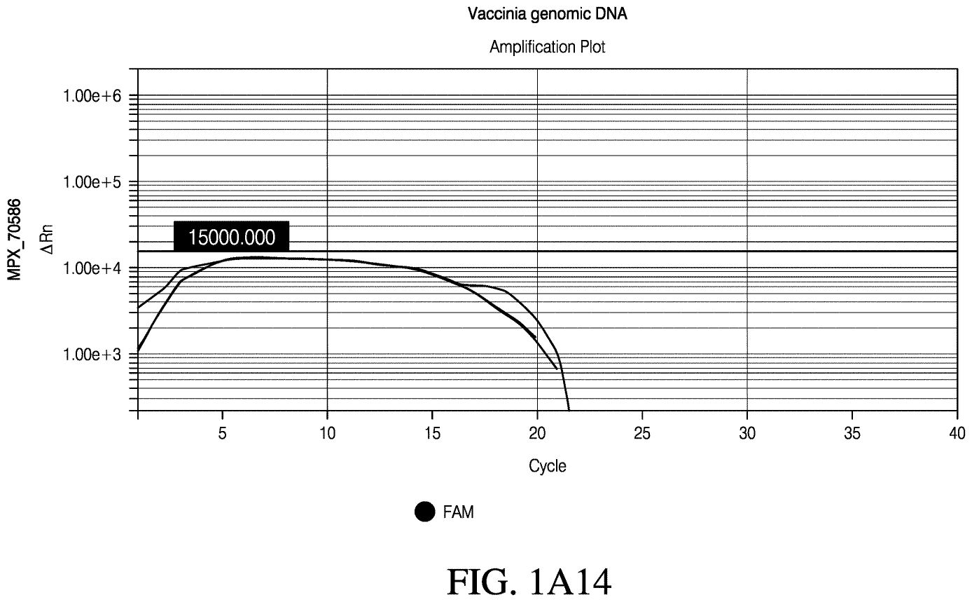

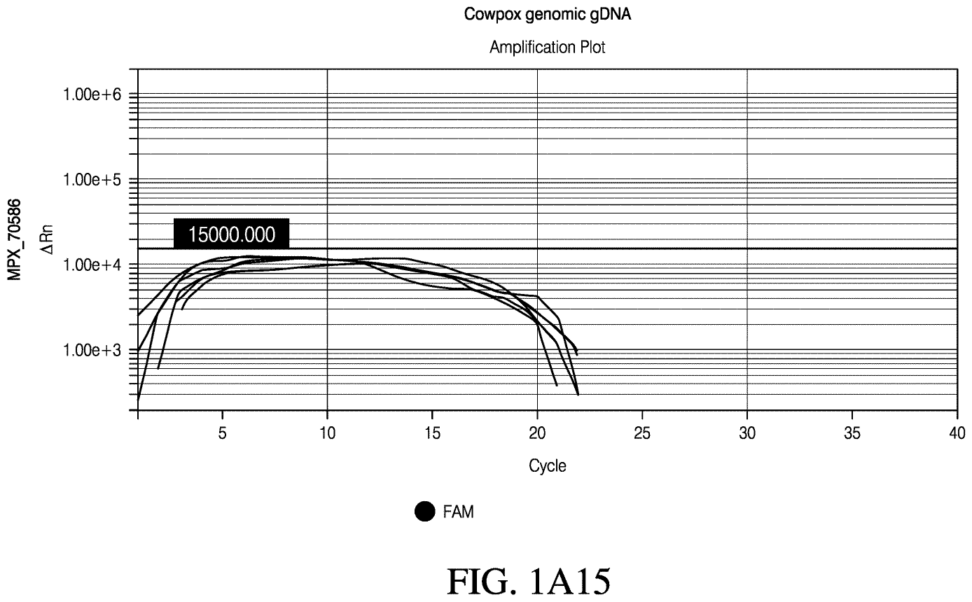

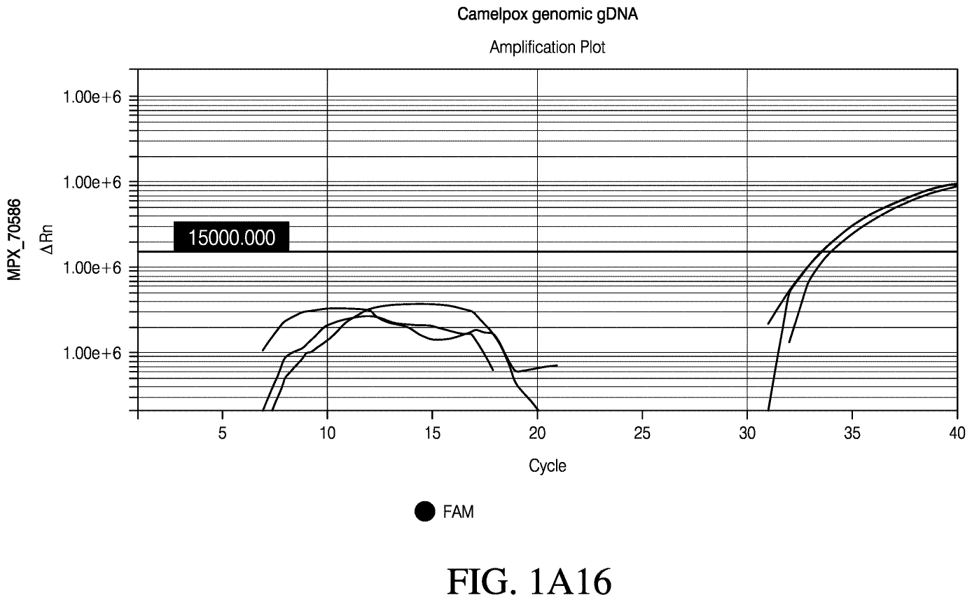

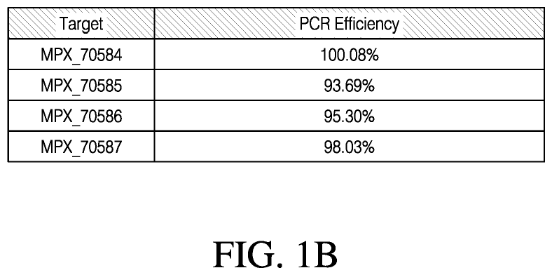

In some other embodiments, the primers and/or probes provided in FIG. 1 can be used to amplify one or more specific target sequences present in the monkeypox virus.

The primer and probe sequences described herein need not have 100% homology/identity to their targets to be effective, though in some embodiments, homology is substantially 100% or exactly 100%. In some embodiments, one or more of the disclosed primer and/or probe sequences have a homology to their respective target of at least about 50%, at least about 60%, at least about 70%, at least about 75%, at least about 80%, at least about 85%, at least about 90%, at least about 95%, at least about 97%, at least about 98%, at least about 99%, at least about 99.9%, or up to substantially 100% or exactly 100%. Some combinations of primers and/or probes may include primers and/or probes each with different homologies to their respective targets, and the homologies may be, for example, within a range with endpoints defined by any two of the foregoing values.

PCR and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as those listed in Table 5, may require less or less extensive thermal cycling than does PCR, or require no thermal cycling. Such isothermal amplification methods are also contemplated for use with the assay compositions, kits, and methods described herein.

TABLE 5
Alternative DNA Amplification Processes

| | |
|---|---|
| LAMP | Loop-mediated isothermal amplification (LAMP) is a single tube technique for the |
| | amplification of DNA. It typically uses 4-6 primers, which form loop structures to |
| | facilitate subsequent rounds of amplification. |
| HDA | Helicase-dependent amplification (HDA) uses the double-stranded DNA unwinding |
| | activity of a helicase to separate strands for in vitro DNA amplification at constant |
| | temperature. |
| RCA | Rolling circle amplification (RCA) starts from a circular DNA template and a short |
| | DNA primer to form a long single-stranded molecule. |
| MDA | Multiple displacement amplification (MDA) is a technique that initiates when |
| | multiple random primers anneal to the DNA template and the polymerase |
| | amplifies DNA at a constant temperature. |
| WGA | When MDA is used to amplify DNA from a whole genome of a cell it is called whole |
| | genome amplification (WGA). |
| RPA | Recombinase polymerase amplification (RPA) is a low-temperature DNA |
| | amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

In some embodiments, different assay products (e.g., amplicons from different viruses) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety. In some embodiments, specific combinations of labels are used to differentiate between monkeypox virus and positive controls. For example, monkeypox virus may be differentiated from general non-variola orthopox virus using different labels such that the label is detectable only in the presence- and amplification-of the associated sequence.

In some embodiments, the amplifying step can include performing qPCR, as that term is defined herein. qPCR is a sensitive and specific method for detecting and optionally quantifying amounts of starting nucleic acid template (e.g., monkeypox viral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Suitable labels are described herein.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, one or more probe sequences are also included in the singleplex reaction volume.

In some array-formatted multiplex embodiments, the reaction volume within the selected tubes, grooves, channels, wells, cavities, sites, or features contains multiple (e.g., 2, 3, 4, 5, 6, etc.) forward and reverse primer sequences and/or multiple probe sequences. For instance, exemplary methods for polymerizing and/or amplifying and detecting nucleic acids suitable for use as described herein are commercially available as TaqMan assays (see, e.g., U.S. U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety).

In some embodiments (e.g., in the well-known and widely used TaqMan™ line of qPCR assays), detecting an amplification product includes detecting a signal emitted by a fluorescent label attached to the 5′ end of a cleavable probe that selectively hybridizes to the amplification product during amplification. The cleavable probe further includes a quencher that quenches the fluorescent label to a ‘baseline’ fluorescence level. The 5′ end of the cleavable probe is cleaved by the polymerase during the extension step, resulting in the separation of the fluorescent label from the quencher and a corresponding increase in fluorescence over baseline. TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. Thus, the oligonucleotide probe includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule and a continuing increase in fluorescence over baseline is measured at each cycle. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

In some embodiments, a passive reference dye, such as ROX™ is included in the reaction mixture. The metric “Rn” is optionally used to track progress of the amplification reaction and to determine the amount of target sequence originally present in the reaction mixture prior to amplification. Rn can be calculated as the fluorescence of the reporter dye divided by the fluorescence of a passive reference dye present in the reaction mixture; i.e., Rn is the reporter signal normalized to the fluorescence signal of the passive reference dye. In some embodiments, Rⁿ is plotted against PCR cycle number. In some embodiments, ARn (calculated as Rn minus the baseline) can be plotted against PCR cycle number. In some embodiments, an amplification plot shows the variation of log (ARn) with PCR cycle number. Ct (threshold cycle) is the intersection between an amplification curve and a threshold line. The lower the Ct value for a given amplification product, the earlier the amplification is detectable and the higher the absolute amount, and the relative concentration, of the corresponding target sequence originally present in the reaction mixture. In some embodiments, cutoffs for Ct are used to determine whether a target sequence was originally present or absent in the reaction mixture prior to amplification. For example, in some embodiments a target sequence is determined to be present if the Ct value is less than or equal to 37.

In various embodiments, a singleplex or multiplex qPCR can include a single TaqMan assay associated with a locus-specific sequence, or multiple TaqMan assays respectively associated with a plurality target sequences in a multiplex format, or multiple TaqMan assays respectively associated with a plurality of loci in a multiplex format. As a non-limiting example, a triplex reaction can include FAM (emission peak at 517 nm), VIC (emission peak at 551 nm), and JUN (emission peak at 617 nm) dyes. As a non-limiting example, a 4-plex reaction can include FAM (emission peak at 517 nm), VIC (emission peak at 551 nm), ABY (emission peak at 580 nm), and JUN (emission peak at 617 nm) dyes. In some embodiments, each dye is associated with one or more target sequences. In some embodiments, one or more dyes are quenched by MGB or a QSY quencher (e.g., QSY7 or QSY21). In some embodiments, each multiplex reaction allows up to 12 targets to be amplified and tracked real-time within a single reaction vessel. In some embodiments, up to 2, 4, 6, 8, 10, or 12 targets are amplified and tracked real-time within a single reaction vessel, using any combination of detectable labels disclosed herein or otherwise known to those of skill in the art. The reporter dyes are optimized to work together with minimal spectral overlap for improved performance. Any combination of dyes described herein can additionally be combined with other dyes (e.g., Mustang Purple (emission peak-654 nm) or one or more Alexa Fluors (e.g., AF647 and AF676)), for use in monitoring fluorescence of a control or for use in a non-emission-spectrum-overlapping 5-plex assay. In addition, the QSY quencher is fully compatible with probes that have minor-groove binder (MGB) quenchers.

Where multiple detection channels are utilized, it is desirable to minimize crosstalk between fluorescence reporters and select reporters that avoid excessive spectral overlap. One example of an assay that includes 5 detection channels incorporates the dyes FAM, ABY, VIC, and JUN, along with Mustang Purple (emission peak of 654 nm) or an appropriate Alexa Fluor, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs. Additional examples of multiplex assays (including related dye compounds, compositions, methods, and kits) are described in International Publication No. WO 2022/020731 A2, published Jan. 27, 2022 and titled “Compositions, Systems and Methods for Biological Analysis Involving Energy Transfer Dye Conjugates and Analytes Comprising the Same”, which is incorporated herein by this reference in its entirety.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy₅.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, AB Y, JUN; FITC; 6-carboxy-4′,5′-dichloro-2′,7′-dimethoxy,fluorescein (JOE)); 6-carboxy-1,4-dichloro-2′,7′-di chloro-fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyHrhodamine), Tetramethylrhodamine (TRITC), WT), Texas Red, Texas Red-X, among others as would be known to those of skill in the art.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30(9): e37), Hayashi et al. (Nucleic Acids Res. 1989 May 11; 17(9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

In some embodiments, the amplicon is labeled by incorporation of or hybridization to labeled primer. In some embodiments, the amplicon is labeled by hybridization to a labeled probe. In some embodiments, the amplicon is labeled by binding of a DNA-binding dye. In some embodiments, the dye may be a single-strand DNA binding dye. In other embodiments, the dye may be a double-stranded DNA binding dye. In other embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In one exemplary embodiment, the labeled amplicon is detected using capillary electrophoresis. In another embodiment, the labeled amplicon is detected using qPCR. In some embodiments, a plurality of different amplicons is formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In some embodiments, the plurality of amplicons can be differentially labeled. In some embodiments, each of the plurality of amplicons produced during amplification is labeled with a different label.

Optionally, in some embodiments, a RNase P assay is included in the kit. Exemplary primers and probes for the RNase P assay can include sequences of a forward primer including SEQ ID NO. 13, a reverse primer including SEQ ID NO. 14, and a probe including SEQ ID NO. 15, although those having skill in the art should appreciate that other RNase-P-specific primers and/or probes could be used.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), the QuantStudio 5 DX RealTime PCR System (QS5DX), or the QuantStudio 7 Flex RealTime PCR System (QS7Flex), from Thermo Fisher Scientific.

In some embodiments, the systems, compositions, methods, and devices used for nucleic acid amplification comprise a “point-of-service” (POS) system. In some embodiments, samples may be collected and/or analyzed at a “point-of-care” (POC) location. In some embodiments, analysis at a POC location typically does not require specialized equipment and has rapid and easy-to-read visual results. In some embodiments, analysis can be performed in the field, in a home setting, and/or by a lay person not having specialized skills. In certain embodiments, for example, the analysis of a small-volume clinical sample may be completed using a POS system in a short period of time (e.g., within hours or minutes).

Optionally, a POS system is utilized at a location that is capable of providing a service (e.g., testing, monitoring, treatment, diagnosis, guidance, sample collection, verification of identity (ID verification), and other services) at or near the site or location of the subject. A service may be a medical service or it may be a non-medical service. In some situations, a POS system provides a service at a predetermined location, such as a subject's home, school, or work, or at a grocery store, a drug store, a community center, a clinic, a doctor's office, a hospital, an outdoor triage tent, a makeshift hospital, a border check point, etc. A POS system can include one or more point of service devices, such as a portable virus/pathogen detector. In some embodiments, a POS system is a point of care system. In some embodiments, the POS system is suitable for use by non-specialized workers or personnel, such as nurses, police officers, civilian volunteers, or the patient.

In certain embodiments, a POC system is utilized at a location at which medical-related care (e.g., treatment, testing, monitoring, diagnosis, counseling, etc.) is provided. A POC may be, e.g., at a subject's home, work, or school, or at a grocery store, a community center, a drug store, a doctor's office, a clinic, a hospital, an outdoor triage tent, a makeshift hospital, a border check point, etc. A POC system is a system which may aid in, or may be used in, providing such medical-related care, and may be located at or near the site or location of the subject or the subject's health care provider (e.g., subject's home, work, or school, or at a grocery store, a community center, a drug store, a doctor's office, a clinic, a hospital, etc.).

In embodiments, a POS system is configured to accept a clinical sample obtained from a subject at the associated POS location. In embodiments, a POS system is further configured to analyze the clinical sample at the POS location. In embodiments, the clinical sample is a small volume clinical sample. In embodiments, the clinical sample is analyzed in a short period of time. In embodiments, the short period of time is determined with respect to the time at which sample analysis began. In embodiments, the short period of time is determined with respect to the time at which the sample was inserted into a device for the analysis of the sample. In embodiments, the short period of time is determined with respect to the time at which the sample was obtained from the subject.

In some embodiments, a POS system or a POC system can include the amplification-based methods, compositions and kits disclosed herein, including any of the described assays and/or assay panels. Such assays are contemplated for use with both thermal cycling amplification workflows and protocols, such as in PCR, as well as isothermal amplification workflows and protocols, such as in LAMP.

In some embodiments, a POS or a POC system comprises self-collection of a biological sample, such as a lesion fluid on a dry swab, lesion fluid swab in viral transport media, lesion fluid on a slide, lesion crust, or lesion roof. In some embodiments, the self-collection may comprise the use of a self-collection kit and/or device, such as a swab or a tube. In some embodiments, the self-collection kit comprises instructions for use, including collection instructions, sample preparation or storage instructions, and/or shipping instructions. For example, the self-collection kit and/or device may be used by an individual, such as lay person, not having specialized skills or medical expertise. In some embodiments, self-collection may be performed by the patient themselves or by any other individual in proximity to the patient, such as but not limited to a parent, a care giver, a teacher, a friend, or other family member.

Notably, in some embodiments, the nucleic acid amplification protocol can be configured for rapid processing (e.g., in less than about 45 minutes) and high throughput, allowing for a minimally invasive method to quickly screen large numbers of individuals in a scalable way. This can be particularly useful to perform asymptomatic testing (e.g., high frequency/widespread testing at schools, workplaces, conventions, sporting events, large social gatherings, etc.) or for epidemiological purposes. The disclosed embodiments can also beneficially provide a lower cost sample collection system and method that enables self-collection (reducing health care professional staffing needs) using a low-cost collection device. This eliminates the requirements for swabs, buffers, virus transmission media (or other specialized transport medium), and the like. The disclosed embodiments also allow for a reduction in Personal Protective Equipment (PPE) requirements and costs. Because the reagents and methods are streamlined (e.g., no precursor nucleic acid purification and/or extraction step), there is a reduced use of nucleic acid preparation plastics which brings a coincident reduction in reagent costs and inventory costs. There is also a beneficial reduced dependence on supply-constrained items, and the compatibility of these methods and kit components with existing equipment improves the flexibility and simplicity of their implementation to the masses. Overall, such embodiments allow for a less expensive assay that can be accomplished more quickly from sample collection through result generation.

Some embodiments relate to kits containing one or more of the primers and/or probes disclosed in Tables 1, 2, 3 and/or 4. In exemplary embodiments, a triplex monkeypox assay kit includes a monkeypox/orthopox virus multiplex assay panel including a solution of MPX primers at about 300 nM, MPX probes at about 250 nM, OPX primers at about 600 nM, OPX probes at about 250 nM, RNase P primers at about 300 nM, and RNase P probes at about 250 nM. In exemplary embodiments, the kit further includes a monkeypox/orthopox virus positive control including a solution of about 10,000 copies/uL.

Optionally, the kit can further include a master mix. In some embodiments, the master mix is a PCR master mix, for instance TaqPath™ BactoPure™ Microbial Detection Master Mix (No ROX™) (Thermo Fisher Scientific, Waltham, MA, Catalog No. A52702). In some embodiments, the kit includes primers, probes, and master mix sufficient to constitute a reaction mixture supporting amplification of at least one or more target regions from monkeypox virus and/or orthopox virus.

In exemplary embodiments, sample preparation is accomplished with the KingFisher™ Flex Purification System (Thermo Fisher Scientific, Catalog No. 5400610) and a MagMAX™ Viral/Pathogen II Nucleic Acid Isolation Kit (Thermo Fisher Scientific, Catalog No. A48383).

In some array-based embodiments, two or more different qPCR assays (each containing a forward primer, a reverse primer, and optionally a probe) are used in a single well, cavity, site or feature of the array and products of each assay can be independently detected. For example, different assay products may be discriminated optically (e.g., using different labels present in components each assay) or using some other suitable method, including as described in U.S. Patent Publication No. 2019/0002963, incorporated by reference herein. In some embodiments, at least one primer of each assay contains an optically detectable label that can be discriminated from the optical label of at least one other assay.

In some embodiments, optimal amplification and detectability for viral genomes is achieved by adding a master mix to the reaction volume prior to amplification. The master mix optionally includes a polymerase, nucleotides, buffers, and salts. In some embodiments (particularly multiplex assays), the reaction volume includes TaqPath BactoPure Microbial Detection Master Mix (No ROX) (Thermo Fisher Scientific, Waltham, MA, Catalog No. A52702).

### EXAMPLES

The following examples may reference specific target nucleic acids, compositions, formulations, and/or process steps. It will be understood, however, that these examples may be modified by using any of the components described elsewhere herein, including by using any of the primers and/or probes described herein.

### Example 1: Screening of Candidate Monkeypox Assays

A. Comparison of Thermo Fisher MPX assay designs. Candidate monkeypox assays were tested with synthetic template specific for respective assay in PCR. Synthetic templates were titrated in PCR from 10 to 107 copies/reaction. In addition, the cross-reactivity with orthopox virus was determined by genomic DNA from representative orthopox viruses as shown. MPX_70585, MPX_70587, MPX_70584 and MPX_70586 are suitable candidates for the MPX panel based on the low Cq values observed in template titration and undetectable cross-reactivity with the three orthopox viruses tested. Referring to Table 1 herein above, the primers and probes are selected from those monkeypox probes as listed. In these assays, primer/probe sets that were employed include: MPX primers/probes sets 26, 34, 40 and 49.

Exemplary results illustrating amplification plots and associated PCR efficiencies are provided in FIGS. 1A and 1B for the candidate monkeypox assays.

B. Comparison of Thermo Fisher OPX assay designs. Two candidate orthopox virus assays, OPX_70588 and OPX_70589 were screened with synthetic templates as well as genomic DNA of three representative orthopox viruses as indicated. Synthetic templates were titrated from 10 to 107 copies/reaction. Both assays were shown to detect strongly with all the templates. OPX_70589 has a slightly better performance with a lower Cq value at the same template concentration. However, both OPX_70588 and OPX_70589 were suitable candidates for the OPX panel.

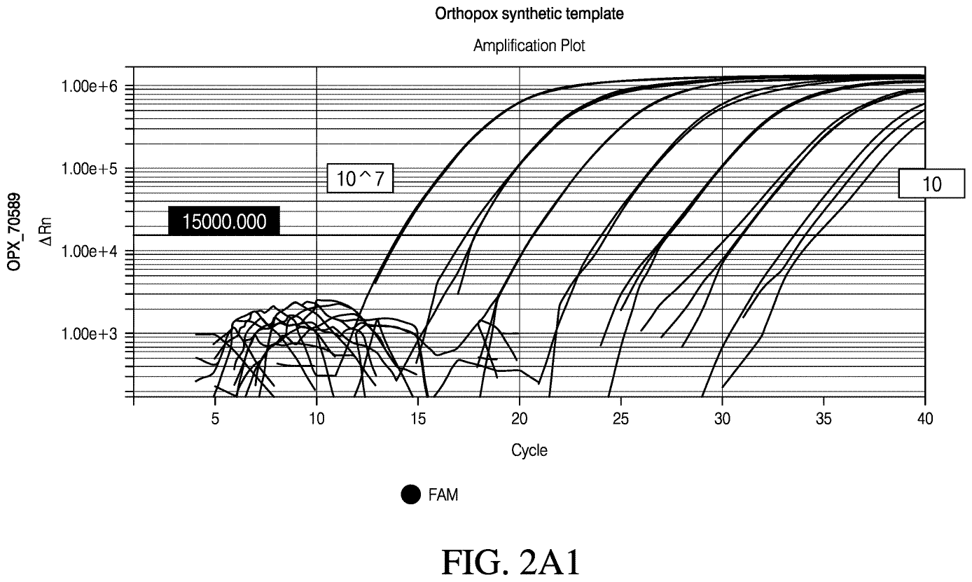

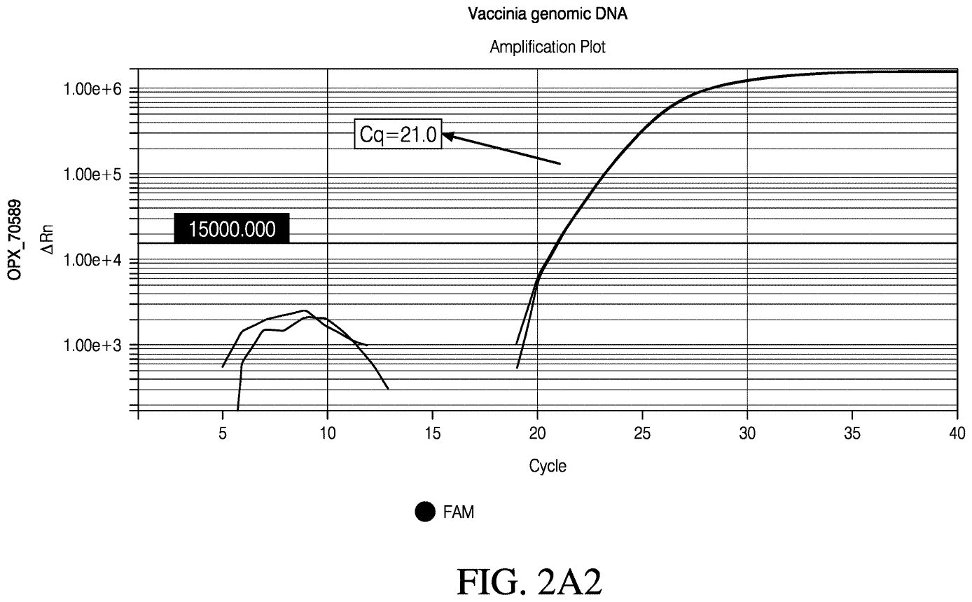

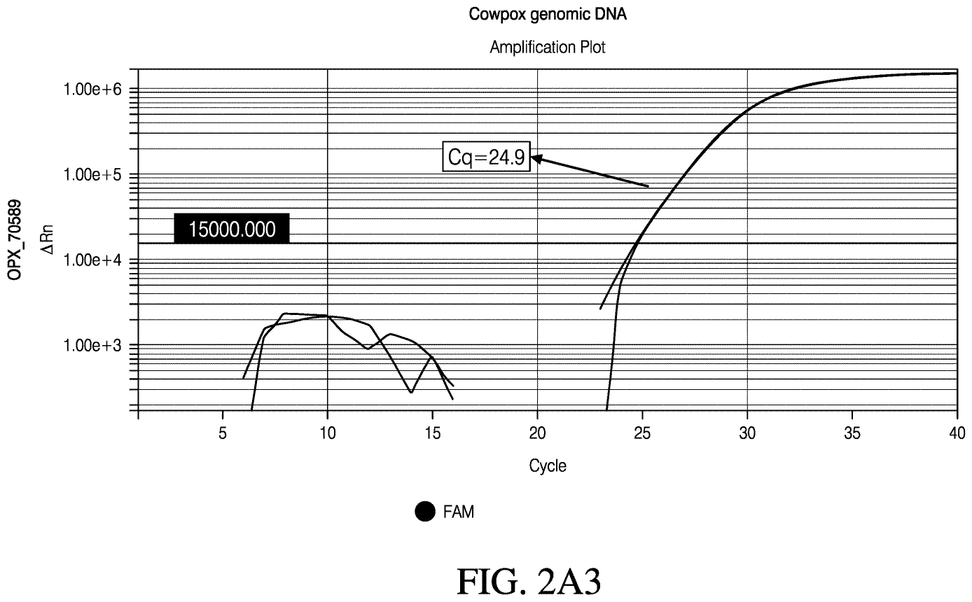

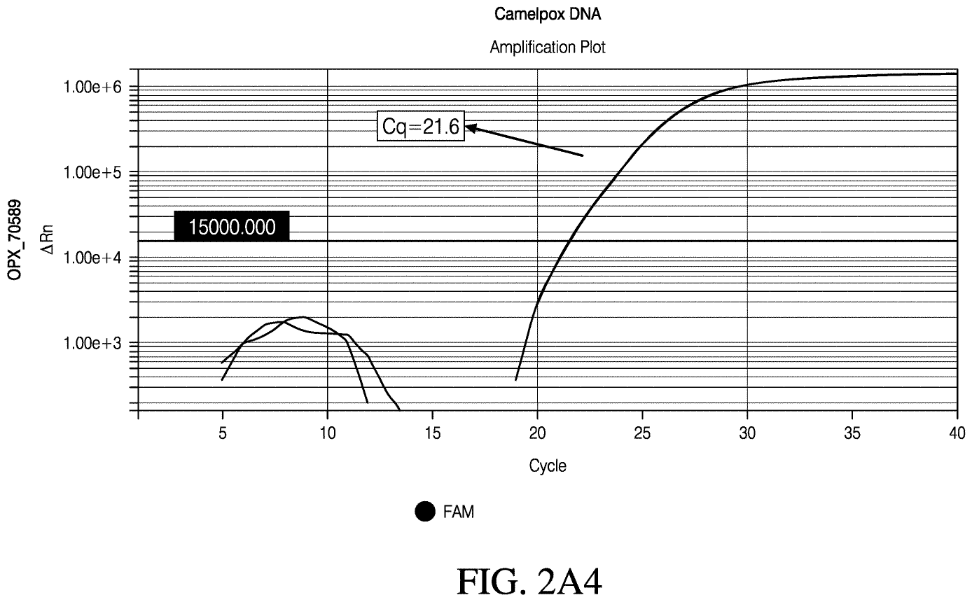

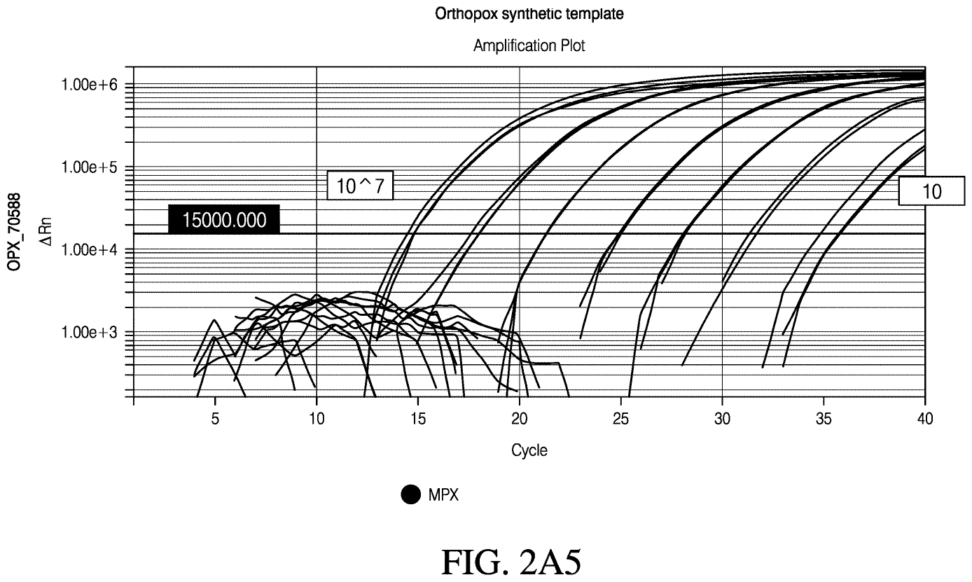

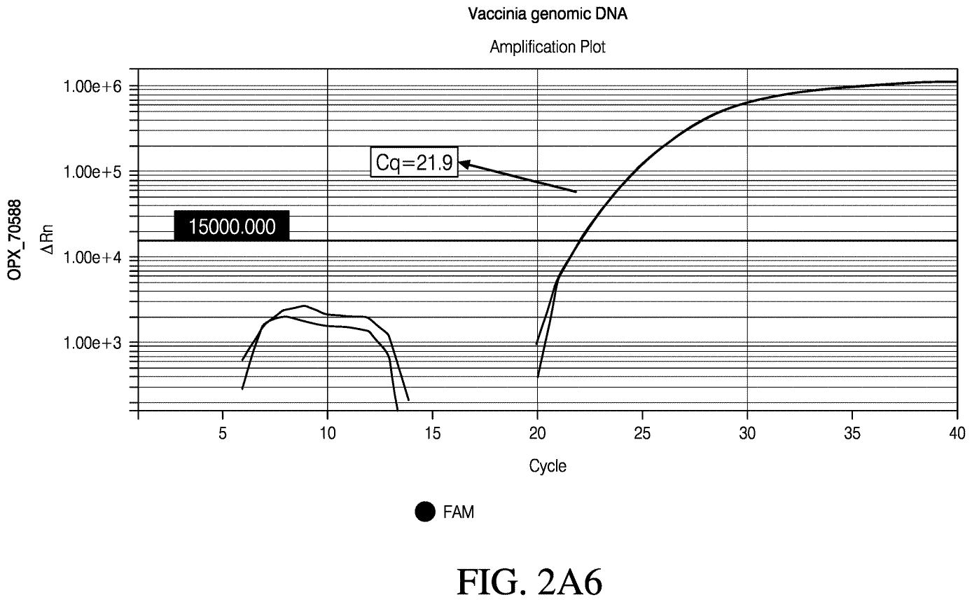

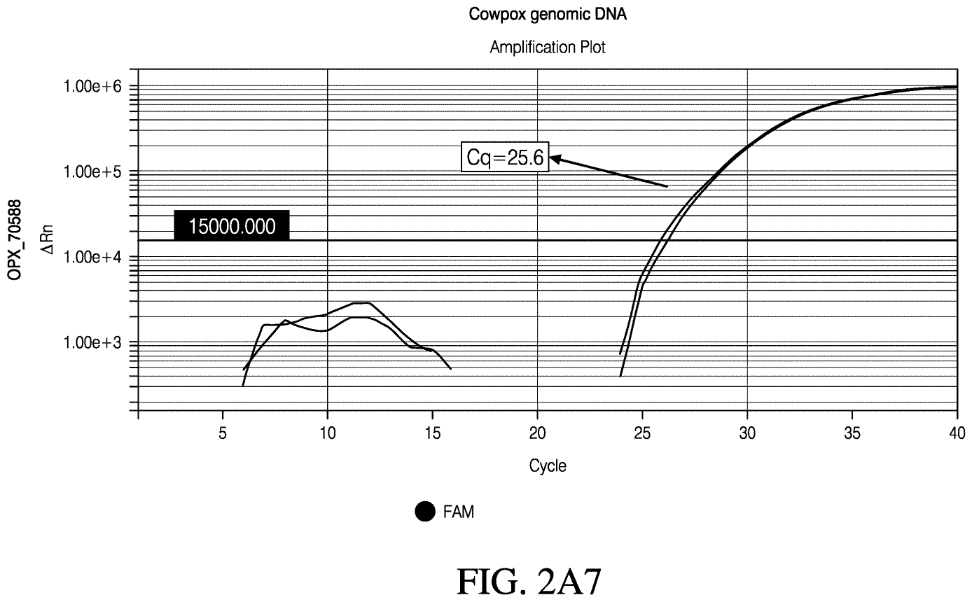

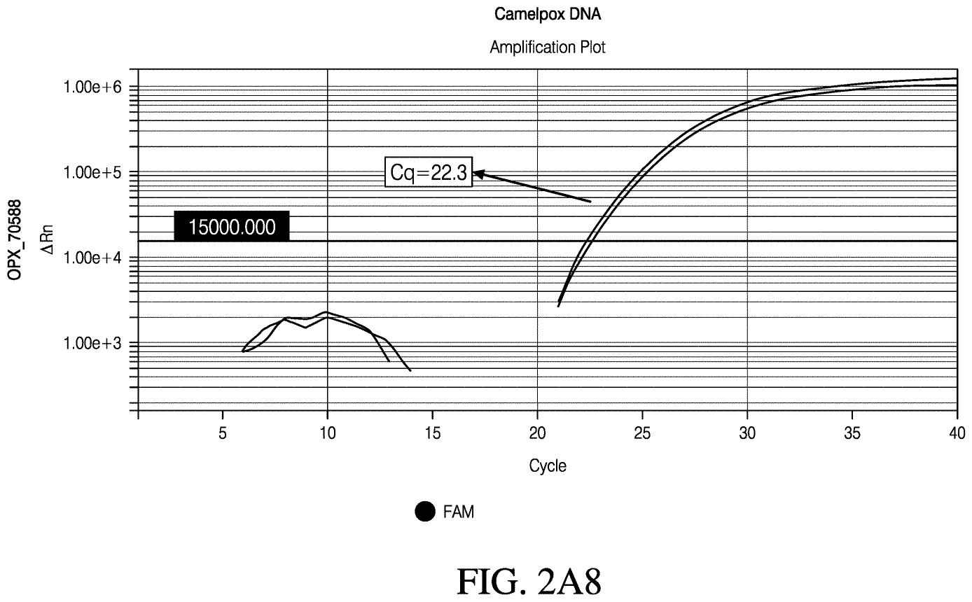

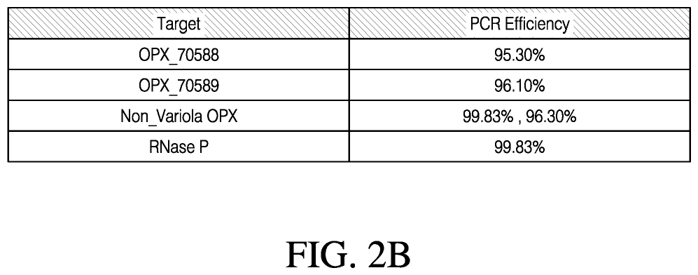

Exemplary results illustrating amplification plots and associated PCR efficiencies are provided in FIGS. 2A and 2B for the candidate orthopox assays.

C. Comparison of OPX_70589 (Thermo Fisher) and non-variola OPX assay (CDC). OPX_70589 was compared with the non-variola OPX assay in a titration experiment with synthetic template. While both assays demonstrated similar PCR efficiency, the non-variola OPX assay showed a better performance with a lower Cq value at the same template concentration. Based on the screening data, OPX_70588, OPX_70589 and the non-variola OPX assay was are suitable candidates for the OPX panel.

Referring to Table 2 herein above, the primers and probes are selected from those OPX probes as listed. In these assays, primer/probe sets that were employed include: OPX primers/probes sets 1, 2 and 3.

Exemplary results illustrating PCR efficiencies are provided in FIG. 2B for the candidate orthopox assays.

### Example 2: Monkeypox/Orthopox Virus DNA Kit and Protocol

Regarding the following Examples 2-9, target oligonucleotides, primers and probes are selected from corresponding Tables 1-4. In these specific assays as described in the following examples, primer/probe sets that were employed include: MPX primers/probes for set 34 from Table 1, OPX primers/probes set 3 from Table 2, primers/probes for RNase P from Table 3.

An exemplary kit and protocol for detecting monkeypox virus from a biological sample via a multiplex assay employs a triplex assay including a three-channel panel with one assay per channel, an MPX assay with an FAM reporter and an MGB quencher, an OPX assay with a VIC reporter and a QSY7 quencher, and an RNase P assay with a JUN reporter and a QSY7 quencher.

Targets and oligonucleotide primers and probes are selected from among those listed in Tables 1~4 herein above.

The monkeypox/orthopox virus DNA kit is a multiplexed polymerase chain reaction (PCR) test intended for the qualitative detection of nucleic acid from monkeypox virus and screening for orthopox viruses in lesion swab specimens in universal transport media (UTM) or viral transport media (VTM) from individuals suspected of monkeypox infection by their healthcare provider. Results are for the identification of monkeypox virus and non-variola orthopox virus DNA, which is generally detectable in lesion swab samples during the acute phase of infection.

The assay kit contains primer and probe sets for the identification of monkeypox viral DNA and also to screen for the other non-variola orthopox viral DNA, including vaccinia, cowpox, monkeypox, and ectromelia viruses at varying concentrations. RNase P, a reference human gene, is selected as an endogenous internal control indicating successful and sufficient specimen collection. Detection of RNase P indicates that human nucleic acid is present and implies that human biological material was collected and successfully extracted and amplified.

Positive results are indicative of the presence of monkeypox or other orthopox viral DNA; clinical correlation with patient history and other diagnostic information is necessary to determine patient infection status.

DNA Kit is a multiplexed real-time PCR testing solution that can detect nucleic acid from monkeypox virus, non-variola orthopox virus targets, and human RNase P in multiple reaction wells.

Each kit includes the following components:

TaqPath™ Monkeypox/Orthopox Virus Multiplex Assay-Multiplexed real-time PCR assay containing primers/probes specific to monkeypox virus, non-variola orthopox virus targets, and human RNase P.

TaqPath™ Monkeypox/Orthopox Virus DNA Positive Control-Positive DNA control that contains templates specific to monkeypox virus, non-variola orthopox virus targets, and human RNase P regions targeted by the assay.

The assay contains primers and probe sets specific to the following targets: monkeypox virus; orthopox virus; and RNase P (human sample collection control).

The TaqPath™ Monkeypox/Orthopox Virus DNA Kit probes contain QSY™ and MGB quenchers, which do not fluoresce. Dyes, quenchers and targets are shown in Table 6.

TABLE 6
Dyes, quenchers, and targets

| Target | Dye | Quencher |
|---|---|---|
| Monkeypox virus | FAM ™ dye | MGB |
| Orthopox virus | VIC ™ dye | QSY7 |
| RNase P | JUN ™ dye | QSY7 |

Results are analyzed using the following software:
Applied Biosystems™ Pathogen Interpretive Software v1.1
SAE Administrator Console Dx v1.0 or SAE Administrator Console Dx v1.2 (for security and audit functions)
Pathogen Interpretive Software 1.0.0 DAT (SAE Profile)
The appropriate assay panel for your instrument:
   MPXOPX-EUA_QS5-9601_1.0.0.zip
   MPXOPX-EUA_QS5Dx-9602_1.0.0.zip
   MPXOPX-EUA_QS7F-384_1.0.0.zip

In the process, the probes anneal to specific target sequences located between unique forward and reverse primers for the following targets: monkeypox virus, orthopox virus, and RNase P (human sample collection control).

During the extension phase of the PCR cycle, the 5′ exonuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity is monitored at each PCR cycle by the real-time PCR instrument.

Other tools, equipment and reagents to be used with the assay kit include software that may be selected from Applied Biosystems™ QuantStudio™ 5 Real-Time PCR System, 96-well, 0.1-mL block, Applied Biosystems™ QuantStudio™ 5 Dx Real-Time PCR System, 96-well, 0.2-mL block, and Applied Biosystems™ QuantStudio™ 7 Flex Real-Time PCR System, 384-well; standard laboratory equipment including freezers, centrifuges, mixers, pipettors and the like, nucleic acid extraction systems; plates; nucleic acid isolation and microbial detection kits, PCR instrumentation and associated plates and consumables.

### Example 3: Example Workflow Using the Kit/Protocol

In an exemplary embodiment, a workflow for detection of Monkeypox virus includes the following steps:
(1) Providing a sample from a patient who is suspected of having a monkeypox/orthopox viral infection;
(2) Perform automated DNA extraction, for example, using the KingFisher™ Flex Magnetic Particle Processor with 96 Deep-Well Head and the MagMAX™ Viral/Pathogen II Nucleic Acid Isolation Kit with 400-μl sample input volume;
(3) Prepare real-time PCR reactions that include the ingredients shown in Table 7, wherein the volumes assume that extracted sample DNA was obtained using an original sample input volume of 400 μL. Table 8 shows the reaction plate volumes for each of the sample, positive and negative controls.

TABLE 7
PCR Ingredients

| | Volume per | Volume for n | Volume for 94 |
|---|---|---|---|
| | DNA sample | DNA samples | DNA samples |
| Component | or control | plus 2 controls | plus |
| TaqPath ™ BactoPure ™ Microbial | 10 μL | 11 × (n + 2) μL | 1056.0 μL |
| Detection Master Mix (No ROX ™) | | | |
| TaqPath ™ Monkeypox/Orthopox | 1 μL | 1.1 × (n + 2) μL | 105.6 μL |
| Virus Multiplex Assay | | | |
| Total reaction mix volume | 11 μL | — | 1,161.6 μL |

TABLE 8
Reaction plate volumes

| | Volume per reaction | | |
|---|---|---|---|
| | DNA | | |
| Component | sample | Positive | Negative |
| Reaction mix (from step 3) | 11 μL | 11 μL | 11 μL |
| Purified sample DNA | 9 μL | — | — |
| (from DNA extraction) | | | |
| Positive control (diluted TaqPath ™ | — | 9 μL | — |
| Monkeypox/Orthopox Virus DNA | | | |
| Positive Control from step 2) | | | |
| Negative control (from DNA extraction) | — | — | 9 μL |
| Total volume | 20 μL | 20 μL | 20 μL |

(4) Perform real-time PCR using a suitable real-time PCR instruments, for example, QuantStudio™ 5 Real-Time PCR Instrument, 96-well, 0.1-mL block; and
(5) Analyze data using suitable software.

### Example 4: Quality Control and Validity of Results

A minimum of one negative control and one positive control must be present for each run. All control wells must pass for the real-time PCR plate to be considered valid (Table 9).

Negative control wells must be run for each extraction that is represented on a real-time PCR plate. All control wells must pass for the real-time PCR plate to be considered valid.

Validation of results is performed automatically by the software based on performance of the positive and negative controls, as shown in Table 10.

TABLE 9
Control samples

| | Monkeypox | Orthopox | | | | |
|---|---|---|---|---|---|---|
| Sample | virus | virus | RNase P | Call | Status | Assessment |
| Positive control | POS | POS | POS | Presence | Passed | REPORT |
| Negative control | NEG | NEG | NEG | Absence | Passed | REPORT |

| | | | | |
|---|---|---|---|---|
| Positive control | All other scenarios | Invalid | Failed | RETEST |
| or negative control | | | | |

TABLE 10
Results interpretation for viral targets in patient samples

| Sample^{[1]} | | | | |
|---|---|---|---|---|
| Monkeypox | Orthopox | RNase | | |
| virus | virus | P | Call | Assessment |
| POS | POS | POS or | Presence | REPORT-Monkeypox virus |
| | | NEG | | and Orthopox virus |
| POS | NEG | POS or | Presence | REPORT-Monkeypox virus |
| | | NEG | | Detected |
| NEG | POS | POS or | Presence | REPORT-Orthopox virus |
| | | NEG | | Detected, Monkeypox virus |
| | | | | Not Detected^{[2]} |
| NEG | NEG | POS | Absence | REPORT-Monkeypox virus |
| | | | | and Orthopox virus |
| | | | | Not Detected |
| NEG | NEG | NEG | Invalid | RETEST |

### Example 5: Limit of Detection (LoD)

This study established the LoD of the TaqPath™ Monkeypox/Orthopox Virus DNA Kit two viral targets: monkeypox virus and orthopox virus.

LoD for each viral target was determined using contrived specimens, synthetic monkeypox virus DNA and genomic vaccinia virus DNA (selected to represent orthopox viruses) spiked at various concentration levels into pooled lesion samples in universal transport medium (UTM). Individual lesion specimens, obtained from the vendor, were confirmed to be negative for monkeypox virus and orthopox virus before pooling. Synthetic monkeypox virus DNA and genomic vaccinia virus DNA were quantitated using Droplet Digital PCR (ddPCR) for use in this study. Sample extraction was performed using the MagMAX™ Viral/Pathogen II Nucleic Acid Isolation Kit on the KingFisher™ Flex Magnetic Particle Processor with 96 Deep-Well Head and each extraction replicate was tested on three PCR instruments: QuantStudio™ 5 Real-Time PCR Instrument (96-well, 0.1-mL block), QuantStudio™ 5 Dx Real-Time PCR Instrument (96-well, 0.2-mL block), and QuantStudio™ 7 Flex Real-Time PCR Instrument (384-well block).

The study was conducted in three phases. Phase I consisted of finding an approximate concentration range for LoD. Phase II refined the concentration test range based on Phase I results. Phase III confirmed the LoD with 20 test replicates. The determined LoD data for each instrument is summarized in Table 11, where the LoD for each viral target is shown in bold.

TABLE 11
LoD: Phase III summary

| | Concentration | QuantStudio ™ 5 | QuantStudio ™ 5 Dx | QuantStudio ™ |
|---|---|---|---|---|
| Viral target | (GCE/mL) | Real-Time PCR | Real-Time PCR | 7 |
| Monkeypox | 100 | 100% (20/20) | 100% (20/20) | 95% (19/20) |
| virus | 50 | 70% (14/20) | 65% (13/20) | 85% (17/20) |
| Orthopox | 300 | 100% (20/20) | 95% (19/20) | 100% (20/20) |
| virus | 250 | 100% (20/20) | 100% (20/20) | 100% (20/20) |

### Example 6: Reactivity (Inclusivity)

In silico analysis was performed to determine reactivity (inclusivity) of the TaqPath™ Monkeypox/Orthopox Virus DNA Kit primer/probe sequences with all known strains/isolates of monkeypox virus (Clades I and II) and non-variola orthopox virus, including vaccinia, cowpox, ectromelia, and camelpox. Based upon BLAST analysis, the TaqPath™ Monkeypox/Orthopox Virus DNA Kit showed 100% homology to >99.6% of monkeypox strains in GISAID and GenBank databases as of 11 Oct. 2022. The impacts of mismatched sequences were assessed and concluded to have minimal impact on the assays' ability to detect monkeypox strains, as mutation locations in assay primers and probes indicate that many of the observed mismatches shall not significantly impact strain detection and the predicted Tm values of assays to mismatched targets are higher than the annealing temperature. The orthopox virus assay was investigated for the predicted inclusivity of non-variola orthopox virus strains (cowpox, camelpox, ectromelia, and vaccinia). As the homologies of the primers or probes are well above 90%, orthopox virus assay primer or probe mismatches are not predicted to impact detection of their respective strains.

Based on the in silico analysis, the TaqPath™ Monkeypox/Orthopox Virus DNA Kit is predicted to perform with high sensitivity on detection of monkeypox strains and non-variola orthopox virus (cowpox, camelpox, ectromelia, and vaccinia strains/isolates) with low failure risk

### Example 8: Clinical Evaluation

A clinical performance evaluation was performed using 30 monkeypox virus contrived positive samples, 30 orthopox virus contrived positive samples, and 30 negative clinical lesion samples using the TaqPath™ Monkeypox/Orthopox Virus DNA Kit. Contrived samples targeted 2×, 3×, and 5×LoD for monkeypox virus and orthopox virus. Each contrived positive sample was prepared using negative lesion samples in transport media (15 samples in UTM and 15 samples in VTM) spiked with synthetic monkeypox virus DNA or genomic vaccinia virus DNA (selected to represent orthopox viruses). All contrived monkeypox virus and orthopox virus positive samples had a detection rate of 100%. All negative lesion samples had a detection rate of 0%. The obtained results were the same QuantStudio™ 5 Real-Time PCR Instrument, QuantStudio™ 5 Dx Real-Time PCR Instrument, and QuantStudio™ 7 Flex Real-Time PCR Instrument and are summarized in Table 13.

TABLE 13
Clinical Results summary

| | | | # of | # of | |
|---|---|---|---|---|---|
| | Concen- | Total | samples | samples | Percent |
| Target | tration | replicates | in VTM | in UTM | agreement^{[1]} |
| Negative | 0× LoD | 30 | 15 | 15 | 100% NPA |
| specimens | | | | | |
| Monkeypox | 2× LoD | 20 | 5 | 15 | 100% PPA |
| virus | 3× LoD | 5 | 5 | 0 | 100% PPA |
| | 5× LoD | 5 | 5 | 0 | 100% PPA |
| | 2× LoD | 20 | 5 | 15 | 100% PPA |
| Orthopox | 3× LoD | 5 | 5 | 0 | 100% PPA |
| virus | 5× LoD | 5 | 5 | 0 | 100% PPA |

^{[1]}NPA = Negative Percent Agreement; PPA = Positive Percent Agreement

### Example 9: C Cutoff Values for Assay Targets

The Ct value for each individual assay was also analyzed. The Pathogen Interpretive Software uses the Ct cutoff values shown in Table 14 for assay targets during interpretation of the results.

TABLE 14
Cₜ cutoff values

| | | Ct cutoff | | |
|---|---|---|---|---|
| | | QuantStudio ™ 5 | QuantStudio ™ 5 Dx | QuantStudio ™ 7 |
| Sample or | | Real-Time PCR | Real-Time PCR | Flex Real-Time PCR |
| Control | Target | Instrument | Instrument | Instrument |

| Positive | RNase P | Valid Cₜ values are ≤36.5 | |
|---|---|---|---|
| control | Viral | Valid Cₜ values are ≤37 | Valid Cₜ values are ≤38 |
| | targets | | |
| Negative | RNase P | Valid Cₜ values are >36.5 | |
| control | Viral | Valid Cₜ values are >37 | Valid Cₜ values are >38 |
| | targets | | |
| Clinical | RNase P | Valid Cₜ values are ≤35 | |
| samples | Viral | Positive Cₜ values are ≤37 | Positive Cₜ values are ≤38 |
| | targets | | |

### Example 10: Example Use of Assay Kit and Procedure

In an exemplary embodiment, monkeypox/orthopox virus detection kit was provided and sample DNA was amplified by PCR with a TaqMan amplification method and a PCR setup as shown in Tables 15-19.

TABLE 15
Components in the kit

| | Concentration (tentative) | |
|---|---|---|
| Components | (Primers/Probe) | Form |
| Monkeypox/Orthopox Virus | (working conc. in PCR) | solution |
| Multiplex Assay Panel | MPX 300/250 nM | |
| | non-variola OPX 600/250 nM | |
| | RNase P 300/250 nM | |
| Monkeypox/Orthopox Virus | (product conc.) | solution |
| Positive Control | 10⁴ copies/uL | |

TABLE 16
Other Components

| | |
|---|---|
| Sample preparation | KingFisher ™ Flex Purification System, |
| | MagMAX ™ Viral/Pathogen II Nucleic Acid Isolation Kit |
| PCR Master Mix | TaqPath ™ BactoPure ™ Microbial Detection Master Mix (No ROX™) |
| PCR instruments | Applied Biosystems ™ QuantStudio ™ 5 Real-Time PCR System, |
| | 96-well, 0.1-mL block, or |
| | Applied Biosystems ™ QuantStudio ™ 5 Dx Real-Time PCR System, |
| | 96-well, 0.2-mL block, or |
| | Applied Biosystems ™ QuantStudio ™ 7 Flex Real-Time PCR System, |
| | 384-well |
| Data analysis software | Pathogen Interpretive Software (EUA) |
| | Design & Analysis (RUO) |

TABLE 17
Assy target Quencher and Reporters

| Assay | Quencher | Reporter |
|---|---|---|
| MPX | MGB | FAM |
| non-variola OPX | QSY7 | VIC |
| RNase P | QSY7 | JUN |

### PCR Amplification Method Employed was TaqMan Amplification

TABLE 18
PCR Setup

| | Sample |
|---|---|
| Component | Volume |
| Master Mix | 10 μL |
| Monkeypox/Orthopox Virus Multiplex Assay Panel | 1 μL |
| Sample | 9 μL |
| Total Volume | 20 μL |

TABLE 19
Thermocycling Conditions

| | | | Number |
|---|---|---|---|
| Step | Temperature | Time | of Cycles |
| UNG incubation | 25° C. | 2 minutes | 1 |
| Activation | 95° C. | 2 minutes | 1 |
| Denaturation | 95° C. | 10 seconds | 40 |
| Anneal/Extension | 60° C. | 30 seconds | |

### EXAMPLE EMBODIMENTS

Provided below is a non-exhaustive list of numbered items reciting certain preferred embodiments:

1. A composition for detecting the presence of monkeypox virus in a biological sample, the composition comprising: a first nucleic acid forward primer, the first nucleic acid forward primer comprising an oligonucleotide including at least one nucleic acid sequence selected from SEQ ID NOs: 16-70; and a first nucleic acid reverse primer, the first nucleic acid reverse primer comprising an oligonucleotide including at least one nucleic acid sequence selected from SEQ ID NOs: 71-125, wherein the first nucleic acid forward primer and the first nucleic acid reverse primer are configured to hybridize to different ends of a first target nucleic acid sequence present in a target region of a genome of the monkeypox virus, to a DNA copy of the first target nucleic acid sequence, or to a complement of the first target nucleic acid sequence or a DNA copy of the complement.

2. The composition of item 1, wherein the first nucleic acid forward primer comprises the oligonucleotide including at least one nucleic acid sequence selected from SEQ ID NOs: 41, 49, 55 and 64; and the first nucleic acid reverse primer comprises the oligonucleotide including at least one nucleic acid sequence selected from SEQ ID NOs: 96, 104, 110 and 119.

3. The composition of any one of items 1 or 2 wherein, the first forward primer includes the oligonucleotide including the at least one nucleic acid sequence having SEQ ID NO. 41.

4. The composition of any one of items 1-3 wherein, the first reverse primer includes the oligonucleotide including the at least one nucleic acid sequence having SEQ ID NO. 96.

5. The composition of any one of items 1 or 2 wherein, the first forward primer includes the oligonucleotide including the at least one nucleic acid sequence having SEQ ID NO. 49.

6. The composition of any one of items 1, 2 or 5 wherein, the first reverse primer includes the oligonucleotide including the at least one nucleic acid sequence having SEQ ID NO. 104.

7. The composition of any one of items 1 or 2 wherein, the first forward primer includes the oligonucleotide including the at least one nucleic acid sequence having SEQ ID NO. 55.

8. The composition of any one of items 1, 2 or 7 wherein, the first reverse primer includes the oligonucleotide including the at least one nucleic acid sequence having SEQ ID NO. 110.

9. The composition of any one of items 1 or 2 wherein, the first forward primer includes the oligonucleotide including the at least one nucleic acid sequence having SEQ ID NO. 64.

10. The composition of any one of items 1, 2 or 9 wherein, the first reverse primer includes the oligonucleotide including the at least one nucleic acid sequence having SEQ ID NO. 119.

11. The composition of any one of the proceeding items, further comprising: a first nucleic acid probe, the first nucleic acid probe comprising an oligonucleotide including at least one nucleic acid sequence selected from SEQ ID NOs: 126-180, wherein the first nucleic acid probe is configured to hybridize to a first target nucleic acid subsequence that is complementary or identical to the first target nucleic acid sequence, or to a DNA copy of the first target nucleic acid subsequence.

12. The composition of item 11, wherein the first nucleic acid probe comprises the oligonucleotide including the at least one nucleic acid sequence selected from SEQ ID NOs. 151, 159, 165 and 174.

13. The composition of item 11 or 12, wherein the first probe comprises the oligonucleotide including the at least one nucleic acid sequence having SEQ ID NO. 151.

14. The composition of item 11 or 12, wherein the first probe comprises the oligonucleotide including the at least one nucleic acid sequence having SEQ ID NO. 159.

15. The composition of item 11 or 12, wherein the first probe comprises the oligonucleotide including the at least one nucleic acid sequence having SEQ ID NO. 165.

16. The composition of item 11 or 12, wherein the first probe comprises the oligonucleotide including the at least one nucleic acid sequence having SEQ ID NO. 174.

17. The composition of any one of items 1-16, wherein the first nucleic acid probe further comprises a detectable label.

18. The composition of item 17, wherein the detectable label is a fluorescent label, and the first nucleic acid probe further comprises a quencher that quenches the detectable label or the fluorescent label.

19. The composition of any one of items 17 or 18, wherein the first nucleic acid probe is labeled at a 5′ end with the detectable label, and at a 3′end with the quencher.

20. The composition of any one of items 1-19 further comprising: a second nucleic acid forward primer comprising an oligonucleotide including at least one nucleic acid sequence selected from SEQ ID Nos: 4, 7 and 10; and a second nucleic acid reverse primer comprising an oligonucleotide including at least one nucleic acid sequence selected from SEQ ID Nos: 5, 8 and 11, wherein the second nucleic acid forward primer and the second nucleic acid reverse primer are configured to hybridize to different ends of a second target nucleic acid sequence present in a target region of a genome of non-variola orthopox virus, to a DNA copy of the second target nucleic acid sequence, or to a complement of the second target nucleic acid sequence or a DNA copy of the complement.

21. The composition of item 20, wherein the second forward primer is the oligonucleotide including the at least one nucleotide sequence having SEQ ID NO. 4, and the second reverse primer is the oligonucleotide including the at least one nucleotide sequence having SEQ ID NO. 5.

22. The composition of item 20, wherein the second forward primer is the oligonucleotide including the at least one nucleotide sequence having SEQ ID NO. 7, and the second reverse primer is the oligonucleotide including the at least one nucleotide sequence having SEQ ID NO. 8.

23. The composition of item 20, wherein the second forward primer is the oligonucleotide including the at least one nucleotide sequence having SEQ ID NO. 10, and the second reverse primer is the oligonucleotide including the at least one nucleotide sequence having SEQ ID NO. 11.

24. The composition of any one of items 1-23, further comprising: a second nucleic acid probe comprising an oligonucleotide including at least one nucleic acid sequence selected from SEQ ID Nos: 6, 9 and 12, wherein the second nucleic acid probe is configured to hybridize to a second target nucleic acid subsequence that is complementary or identical to the second target nucleic acid sequence, or to a DNA copy of the second target nucleic acid subsequence.

25. The composition of item 24, wherein the second nucleic acid probe comprises the oligonucleotide including the at least one nucleic acid sequence having SEQ ID NO. 6.

26. The composition of item 24, wherein the second nucleic acid probe comprises the oligonucleotide including the at least one nucleic acid sequence having SEQ ID NO. 9.

27. The composition of item 24, wherein the second nucleic acid probe comprises the oligonucleotide including the at least one nucleic acid sequence having SEQ ID NO. 12.

28. The composition of any one of items 24-27, wherein the second nucleic acid probe further comprises a detectable label.

29. The composition of item 28, wherein the detectable label is a fluorescent label, and the second nucleic acid probe further comprises a quencher that quenches the fluorescent label.

30. The composition of any one of items 28 or 29, wherein the second nucleic acid probe is labeled at a 5′ end with the detectable label, and at a 3′end with the quencher.

31. The composition of any one of items 1-30, further comprising: a third nucleic acid forward primer comprising an oligonucleotide including a nucleic acid sequence having SEQ ID NO: 13; and a third nucleic acid reverse primer comprising an oligonucleotide including a nucleic acid sequence having SEQ ID NO: 14, wherein the third nucleic acid forward primer and the third nucleic acid reverse primer are configured to hybridize to different ends of a third target nucleic acid sequence present in a target region of a genome of human ribonuclease P, to a DNA copy of the third target nucleic acid sequence, or to a complement of the third target nucleic acid sequence or a DNA copy of the complement.

32. The composition of item 31, further comprising: a third nucleic acid probe comprising an oligonucleotide including a nucleic acid sequence having SEQ ID NO: 15, wherein the third nucleic acid probe is configured to hybridize to a third target nucleic acid subsequence that is complementary or identical to the third target nucleic acid sequence, or to a DNA copy of the third target nucleic acid subsequence.

33. The composition of item 32, wherein the third nucleic acid probe further comprises a detectable label.

34. The composition of item 33, wherein the detectable label is a fluorescent label, and the third nucleic acid probe further comprises a quencher that quenches the fluorescent label.

35. The composition of any one of items 33 or 34, wherein the third nucleic acid probe is labeled at a 5′ end with the detectable label, and at a 3′end with the quencher.

36. The composition of any one of items 1-35, further comprising: the biological sample, a polymerase, a buffer, and dNTPs.

37. The composition of any one of items 1-36, wherein the biological sample is a nucleic acid sample, and the nucleic acid sample is a DNA sample.

38. The composition of any one of items 1-37, wherein the biological sample is selected from the group consisting of a lesion fluid on a dry swab, a lesion fluid swab in viral transport media, lesion fluid on a slide, lesion crust, lesion roof, a saliva sample, a buccal sample, a nasal sample, a nasal pharyngeal sample, a blood sample, a urine sample, and a semen sample.

39. The composition of any one of items 1-38, wherein the biological sample is a human sample.

40. The composition of any one of items 1-38, wherein the biological sample is a non-human sample.

41. A kit for detecting monkeypox virus nucleic acid in a biological sample, comprising: one or more polymerase chain reaction (PCR) reagents, wherein the one or more PCR reagents comprise the composition according to any one of items 1-40.

42. The kit of item 41, further comprising: a PCR master mix.

43. The kit of item 41 or 42, wherein at least one component of the kit is dried or freeze dried.

44. The kit of any one of items 41-43, further comprising: an array of PCR assays, wherein each of the PCR assays is situated in a different locus of the array, and the different locus include a well, a channel, a groove, a cavity, a site, or a feature formed on a surface of the array.

45. A method for the detection of monkeypox virus in a biological sample, the method comprising: providing a reaction mixture containing the biological sample and the composition according to any one of items 1-40; and subjecting the reaction mixture to reaction conditions suitable to amplify a target monkeypox virus nucleic acid sequence present in the biological sample prior to amplification, the target monkeypox virus nucleic acid sequence being the first target nucleic acid sequence; and detecting the amplified target monkeypox virus nucleic acid sequence.

46. The method of item 45, wherein the subjecting the reaction mixture to reaction conditions suitable to amplify the target monkeypox virus nucleic acid sequence includes forming one or more amplicons via polymerase chain reaction (PCR).

47. The method of any one of items 45 or 46, wherein the detecting of the amplified target monkeypox virus nucleic acid includes monitoring fluorescence produced during the PCR.

48. The method of any one of items 45-47, further including determining an amount of monkeypox virus nucleic acid present in the biological sample.

49. The method of any one of items 45-48, further comprising: analyzing a positive control and/or a negative control are analyzed in conjunction with the biological sample.

50. The method of item 49, wherein the positive control is a synthetic oligonucleotide comprising a monkeypox virus sequence, a non-variola orthopox virus sequence, and an endogenous DNA or RNA sequence.

51. The method of item 50, wherein the endogenous DNA or RNA sequence is a human RNaseP sequence.

52. The method of any one of items 45-51, further comprising: identifying the monkeypox virus as a variant or as reference form.

53. The method of any one of items 45-52, wherein the detecting of the amplified target monkeypox virus nucleic acid sequence occurs during and/or after the subjecting of the reaction mixture to reaction conditions suitable to amplify the target monkeypox virus nucleic acid sequence.

54. The method of any one of items 45-53, further including diagnosing a monkeypox infection in an organism from which the biological sample was obtained.

55. The method of item 54, wherein the organism is a human subject.

56. The method of any one of items 45-55, wherein the subjecting the reaction mixture to reaction conditions suitable to amplify the target monkeypox virus nucleic acid sequence includes performing a loop-mediated isothermal amplification (LAMP).

57. The method of any one of items 45-56, wherein the method excludes purifying or extracting a nucleic-acid-containing portion from the biological sample.

58. The method of any one of items 45-57, wherein the biological sample is an unpurified sample.

59. The method of any one of items 45-58, further comprising: heating the biological sample for a period of time sufficient to inactivate nucleases within the biological sample and/or to rupture eukaryotic cells, denature viral capsids, and/or disrupt a membrane portion of enveloped virions therein.

60. The method of any one of items 57-59, further comprising: heating the unpurified sample to a temperature at or above about 80° C., preferably at or above about 90° C., more preferably at or above about 95° C.

61. The method of item 60, wherein the unpurified sample is heated for at least 5, 10, 15, 20, 25, 30, 35, or 40 minutes or for any range of time formed by an upper and lower bound selected therefrom.

62. The method of any one of items 60 or 61, further comprising: combining the heat-treated sample with a lysis buffer.

63. The method of item 62, wherein the lysis buffer comprises a nucleic-acid-amenable buffer and a detergent and/or emulsifier.

64. The method of any one of items 62-63, wherein the lysis buffer comprises a combination of TBE buffer and a polysorbate-type nonionic surfactant, such as Tween-20.

65. The method of any one of items 45-64, wherein the biological sample comprises a pooled-subject sample.

66. The method of any one of items 45-64, wherein detecting monkeypox virus within the biological sample occurs in less than about 3 hours from the time the biological sample is received, preferably less than about 2 hours.

67. The method of any one of items 45-56 and 65-66, wherein the providing of the reaction mixture includes heating the biological sample for 15-45 minutes, preferably about 30 minutes, at 95° C.; mixing the heated biological sample with a lysis solution to form a volume of probative template; creating a nucleic acid amplification reaction mixture comprising at least a portion of the volume of probative template, the composition according to items 1-40, and a nucleic acid polymerase.

68. The method of any one of items 45-67, further comprising: receiving the biological sample.

69. The method of item 68, wherein the receiving the biological sample comprises receiving a sample collection device or other container comprising the biological sample.

70. The method of item 69, wherein the sample collection device is a sealable tube.

71. The method of any one of items 68-71, wherein the biological sample is received following self-collection of the biological sample by a subject.

72. The method of any one of items 68-71, wherein the receiving of the biological sample comprises receiving a raw saliva sample.

73. The method of any one of items 67-71, further comprising: equilibrating the heated biological sample to room temperature prior to mixing the heated sample with the lysis solution.

74. The method of any one of items 67-74, wherein a sample collection device is used to receive and heat the biological sample.

75. A composition for multiplex detection of viral monkeypox nucleic acid and viral orthopox nucleic acid, the composition comprising: a first primer-probe set including a first forward primer and a first reverse primer each configured to hybridize to different ends of a first target nucleic acid sequence present in a target region of monkeypox viral DNA, to a DNA copy of the first target nucleic acid sequence, or to a complement of the first target nucleic acid sequence or a DNA copy of the complement, and a first probe configured to hybridize to a first target nucleic acid subsequence that is complementary or identical to the first target nucleic acid sequence, or to a DNA copy of the first target nucleic acid subsequence, wherein the first primer-probe set includes at least one nucleic acid sequence selected from SEQ ID NOs: 16-180; a second primer-probe set including a second forward primer and a second reverse primer each configured to hybridize to different ends of a second target sequence present in a target region of orthopox viral DNA, to a DNA copy of the second target nucleic acid sequence, or to a complement of the second target nucleic acid sequence or a DNA copy of the complement, and a second probe configured to hybridize to a second target nucleic acid subsequence that is complementary or identical to the second target nucleic acid sequence, or to a DNA copy of the second target nucleic acid subsequence, wherein the second primer-probe set includes at least one nucleic acid sequence selected from SEQ ID NOs: 4-12.

76. The composition according to item 75, wherein the first primer-probe set includes the at least one nucleic acid sequence selected from SEQ ID NOs: 41, 49, 55, 64, 96, 104, 110, 119, 151, 159, 165 and 174.

77. The composition of any one of items 75 or 76 wherein, the first primer-probe set includes the at least one nucleic acid sequence having SEQ ID NO. 41.

78. The composition of any one of items 75-77, wherein the first primer-probe set further includes the at least one nucleic acid sequence having SEQ ID NO. 96.

79. The composition of any one of items 75-78, wherein the first primer-probe set further includes the at least one nucleic acid sequence having SEQ ID NO. 151.

80. The composition of any one of items 75 or 76, wherein the first primer-probe set includes the at least one nucleic acid sequence having SEQ ID NO. 49.

81. The composition of any one of items 75, 76 or 80, wherein the first primer-probe set further includes the at least one nucleic acid sequence having SEQ ID NO. 104.

82. The composition of any one of items 75, 76, 80 or 81, wherein the first primer-probe set further includes the at least one nucleic acid sequence having SEQ ID NO. 159.

83. The composition of any one of items 75 or 76, wherein the first primer-probe set includes the at least one nucleic acid sequence having SEQ ID NO. 55.

84. The composition of any one of items 75, 76 or 83, wherein the first primer-probe set further includes the at least one nucleic acid sequence having SEQ ID NO. 110.

85. The composition of any one of items 75, 76, 83 or 84, wherein the first primer-probe set further includes the at least one nucleic acid sequence having SEQ ID NO. 165.

86. The composition of any one of items 75 or 76, wherein the first primer-probe set includes the at least one nucleic acid sequence having SEQ ID NO. 64.

87. The composition of any one of items 75, 76 or 86, wherein the first primer-probe set further includes the at least one nucleic acid sequence having SEQ ID NO. 119.

88. The composition of any one of items 75, 76, 86 or 87 wherein, the first primer-probe set further includes the at least one nucleic acid sequence having SEQ ID NO. 174.

89. The composition of any one of items 75-88, wherein the second primer-probe set includes the at least one nucleic acid sequence having SEQ ID NOs. 4-6.

90. The composition of any one of items 75-88, wherein the second primer-probe set includes the at least one nucleic acid sequence having SEQ ID NOs. 7-9.

91. The composition of any one of items 75-88, wherein the second primer-probe set includes the at least one nucleic acid sequence having SEQ ID NOs. 10-12.

92. The composition according to any one of items 75-91, further comprising: a third primer-probe set including a third forward primer and a third reverse primer each configured to hybridize to different ends of a third target nucleic acid sequence present in a target region of human ribonuclease P, and a third probe configured to hybridize to a third target nucleic acid subsequence that is complementary or identical to the third target nucleic acid sequence, or to a DNA copy of the third target nucleic acid subsequence, wherein the third primer-probe set includes at least one nucleic acid sequence selected from SEQ ID NOs: 13-15.

93. The composition of any one of items 75-92, wherein the first nucleic acid probe, the second nucleic acid probe and the third nucleic acid probe each further comprises a different detectable label.

94. The composition of item 93, wherein the detectable label is a fluorescent label, and the first nucleic acid probe, the second nucleic acid probe and the third nucleic acid probe each further comprises a different quencher that quenches the respective detectable label or the respective fluorescent label.

95. The composition of any one of items 93 or 94, wherein the first nucleic acid probe, the second nucleic acid probe and the third nucleic acid probe are each labeled at a 5′ end with the detectable label, and at a 3′end with the quencher.

96. The composition of any one of items 75-95, further comprising: the biological sample, a polymerase, a buffer, and dNTPs.

97. The composition of any one of items 75-96, wherein the biological sample is a nucleic acid sample, and the nucleic acid sample is a DNA sample.

98. The composition of any one of items 75-96, wherein the biological sample is selected from the group consisting of a lesion fluid on a dry swab, a lesion fluid swab in viral transport media, lesion fluid on a slide, lesion crust, lesion roof, a saliva sample, a buccal sample, a nasal sample, a nasal pharyngeal sample, a blood sample, a urine sample, and a semen sample.

99. The composition of any one of items 75-98, wherein the biological sample is a human sample.

100. The composition of any one of items 75-98 wherein the biological sample is a non-human sample.

### CONCLUSION

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatuses disclosed herein may be made without departing from the scope of the disclosure or of the invention. Thus, while various aspects and embodiments have been disclosed herein, other aspects and embodiments are contemplated. All changes that come within the meaning and range of equivalency of the claims are to be embraced within their scope.

What is claimed is:

1. A composition for detecting the presence of monkeypox virus in a biological sample, the composition comprising:
a first nucleic acid forward primer, the first nucleic acid forward primer comprising an oligonucleotide including at least one nucleic acid sequence selected from SEQ ID NOs: 16-70; and
a first nucleic acid reverse primer, the first nucleic acid reverse primer comprising an oligonucleotide including at least one nucleic acid sequence selected from SEQ ID NOs: 71-125,
wherein the first nucleic acid forward primer and the first nucleic acid reverse primer are configured to hybridize to different ends of a first target nucleic acid sequence present in a target region of a genome of the monkeypox virus, to a DNA copy of the first target nucleic acid sequence, or to a complement of the first target nucleic acid sequence or a DNA copy of the complement.

2. The composition of claim 1, wherein
the first nucleic acid forward primer comprises the oligonucleotide including at least one nucleic acid sequence selected from SEQ ID NOs: 41, 49, 55 and 64; and
the first nucleic acid reverse primer comprises the oligonucleotide including at least one nucleic acid sequence selected from SEQ ID NOs: 96, 104, 110 and 119.

3. The composition of any one of claim 1 or 2 wherein, the first forward primer includes the oligonucleotide including the at least one nucleic acid sequence having SEQ ID NO. 41.

4. The composition of any one of claims 1-3 wherein, the first reverse primer includes the oligonucleotide including the at least one nucleic acid sequence having SEQ ID NO. 96.

5. The composition of any one of claim 1 or 2 wherein, the first forward primer includes the oligonucleotide including the at least one nucleic acid sequence having SEQ ID NO. 49.

6. The composition of any one of claim 1, 2 or 5 wherein, the first reverse primer includes the oligonucleotide including the at least one nucleic acid sequence having SEQ ID NO. 104.

7. The composition of any one of claim 1 or 2 wherein, the first forward primer includes the oligonucleotide including the at least one nucleic acid sequence having SEQ ID NO. 55.

8. The composition of any one of claim 1, 2 or 7 wherein, the first reverse primer includes the oligonucleotide including the at least one nucleic acid sequence having SEQ ID NO. 110.

9. The composition of any one of claim 1 or 2 wherein, the first forward primer includes the oligonucleotide including the at least one nucleic acid sequence having SEQ ID NO. 64.

10. The composition of any one of claim 1, 2 or 9 wherein, the first reverse primer includes the oligonucleotide including the at least one nucleic acid sequence having SEQ ID NO. 119.

11. The composition of any one of the proceeding claims, further comprising:
a first nucleic acid probe, the first nucleic acid probe comprising an oligonucleotide including at least one nucleic acid sequence selected from SEQ ID NOs: 126-180,
wherein the first nucleic acid probe is configured to hybridize to a first target nucleic acid subsequence that is complementary or identical to the first target nucleic acid sequence, or to a DNA copy of the first target nucleic acid subsequence.

12. The composition of claim 11, wherein the first nucleic acid probe comprises the oligonucleotide including the at least one nucleic acid sequence selected from SEQ ID NOs. 151, 159, 165 and 174.

13. The composition of claim 11 or 12, wherein the first probe comprises the oligonucleotide including the at least one nucleic acid sequence having SEQ ID NO. 151.

14. The composition of claim 11 or 12, wherein the first probe comprises the oligonucleotide including the at least one nucleic acid sequence having SEQ ID NO. 159.

15. The composition of claim 11 or 12, wherein the first probe comprises the oligonucleotide including the at least one nucleic acid sequence having SEQ ID NO. 165.

16. The composition of claim 11 or 12, wherein the first probe comprises the oligonucleotide including the at least one nucleic acid sequence having SEQ ID NO. 174.

17. The composition of any one of claims 1-16, wherein the first nucleic acid probe further comprises a detectable label.

18. The composition of claim 17, wherein the detectable label is a fluorescent label, and the first nucleic acid probe further comprises a quencher that quenches the detectable label or the fluorescent label.

19. The composition of any one of claim 17 or 18, wherein the first nucleic acid probe is labeled at a 5′ end with the detectable label, and at a 3′end with the quencher.

20. The composition of any one of claims 1-19 further comprising:
a second nucleic acid forward primer comprising an oligonucleotide including at least one nucleic acid sequence selected from SEQ ID Nos: 4, 7 and 10; and
a second nucleic acid reverse primer comprising an oligonucleotide including at least one nucleic acid sequence selected from SEQ ID Nos: 5, 8 and 11,
wherein the second nucleic acid forward primer and the second nucleic acid reverse primer are configured to hybridize to different ends of a second target nucleic acid sequence present in a target region of a genome of non-variola orthopox virus, to a DNA copy of the second target nucleic acid sequence, or to a complement of the second target nucleic acid sequence or a DNA copy of the complement.

21. The composition of claim 20, wherein
the second forward primer is the oligonucleotide including the at least one nucleotide sequence having SEQ ID NO. 4, and
the second reverse primer is the oligonucleotide including the at least one nucleotide sequence having SEQ ID NO. 5.

22. The composition of claim 20, wherein
the second forward primer is the oligonucleotide including the at least one nucleotide sequence having SEQ ID NO. 7, and
the second reverse primer is the oligonucleotide including the at least one nucleotide sequence having SEQ ID NO. 8.

23. The composition of claim 20, wherein
the second forward primer is the oligonucleotide including the at least one nucleotide sequence having SEQ ID NO. 10, and
the second reverse primer is the oligonucleotide including the at least one nucleotide sequence having SEQ ID NO. 11.

24. The composition of any one of claims 1-23, further comprising:
a second nucleic acid probe comprising an oligonucleotide including at least one nucleic acid sequence selected from SEQ ID Nos: 6, 9 and 12,
wherein the second nucleic acid probe is configured to hybridize to a second target nucleic acid subsequence that is complementary or identical to the second target nucleic acid sequence, or to a DNA copy of the second target nucleic acid subsequence.

25. The composition of claim 24, wherein the second nucleic acid probe comprises the oligonucleotide including the at least one nucleic acid sequence having SEQ ID NO. 6.

26. The composition of claim 24, wherein the second nucleic acid probe comprises the oligonucleotide including the at least one nucleic acid sequence having SEQ ID NO. 9.

27. The composition of claim 24, wherein the second nucleic acid probe comprises the oligonucleotide including the at least one nucleic acid sequence having SEQ ID NO. 12.

28. The composition of any one of claims 24-27, wherein the second nucleic acid probe further comprises a detectable label.

29. The composition of claim 28, wherein the detectable label is a fluorescent label, and the second nucleic acid probe further comprises a quencher that quenches the fluorescent label.

30. The composition of any one of claim 28 or 29, wherein the second nucleic acid probe is labeled at a 5′ end with the detectable label, and at a 3′end with the quencher.

31. The composition of any one of claims 1-30, further comprising:
a third nucleic acid forward primer comprising an oligonucleotide including a nucleic acid sequence having SEQ ID NO: 13; and
a third nucleic acid reverse primer comprising an oligonucleotide including a nucleic acid sequence having SEQ ID NO: 14,
wherein the third nucleic acid forward primer and the third nucleic acid reverse primer are configured to hybridize to different ends of a third target nucleic acid sequence present in a target region of a genome of human ribonuclease P, to a DNA copy of the third target nucleic acid sequence, or to a complement of the third target nucleic acid sequence or a DNA copy of the complement.

32. The composition of claim 31, further comprising:
a third nucleic acid probe comprising an oligonucleotide including a nucleic acid sequence having SEQ ID NO: 15, wherein the third nucleic acid probe is configured to hybridize to a third target nucleic acid subsequence that is complementary or identical to the third target nucleic acid sequence, or to a DNA copy of the third target nucleic acid subsequence.

33. The composition of claim 32, wherein the third nucleic acid probe further comprises a detectable label.

34. The composition of claim 33, wherein the detectable label is a fluorescent label, and the third nucleic acid probe further comprises a quencher that quenches the fluorescent label.

35. The composition of any one of claim 33 or 34, wherein the third nucleic acid probe is labeled at a 5′ end with the detectable label, and at a 3′end with the quencher.

36. The composition of any one of claims 1-35, further comprising:
the biological sample, a polymerase, a buffer, and dNTPs.

37. The composition of any one of claims 1-36, wherein the biological sample is a nucleic acid sample, and the nucleic acid sample is a DNA sample.

38. The composition of any one of claims 1-37, wherein the biological sample is selected from the group consisting of a lesion fluid on a dry swab, a lesion fluid swab in viral transport media, lesion fluid on a slide, lesion crust, lesion roof, a saliva sample, a buccal sample, a nasal sample, a nasal pharyngeal sample, a blood sample, a urine sample, and a semen sample.

39. The composition of any one of claims 1-38, wherein the biological sample is a human sample.

40. The composition of any one of claims 1-38, wherein the biological sample is a non-human sample.

41. A kit for detecting monkeypox virus nucleic acid in a biological sample, comprising:
one or more polymerase chain reaction (PCR) reagents, wherein the one or more PCR reagents comprise the composition according to any one of claims 1-40.

42. The kit of claim 41, further comprising:
a PCR master mix.

43. The kit of claim 41 or 42, wherein at least one component of the kit is dried or freeze dried.

44. The kit of any one of claims 41-43, further comprising:
an array of PCR assays, wherein each of the PCR assays is situated in a different locus of the array, and
the different locus include a well, a channel, a groove, a cavity, a site, or a feature formed on a surface of the array.

45. A method for the detection of monkeypox virus in a biological sample, the method comprising:
providing a reaction mixture containing the biological sample and the composition according to any one of claims 1-40; and
subjecting the reaction mixture to reaction conditions suitable to amplify a target monkeypox virus nucleic acid sequence present in the biological sample prior to amplification, the target monkeypox virus nucleic acid sequence being the first target nucleic acid sequence; and
detecting the amplified target monkeypox virus nucleic acid sequence.

46. The method of claim 45, wherein the subjecting the reaction mixture to reaction conditions suitable to amplify the target monkeypox virus nucleic acid sequence includes forming one or more amplicons via polymerase chain reaction (PCR).

47. The method of any one of claim 45 or 46, wherein the detecting of the amplified target monkeypox virus nucleic acid includes monitoring fluorescence produced during the PCR.

48. The method of any one of claims 45-47, further including determining an amount of monkeypox virus nucleic acid present in the biological sample.

49. The method of any one of claims 45-48, further comprising:
analyzing a positive control and/or a negative control are analyzed in conjunction with the biological sample.

50. The method of claim 49, wherein the positive control is a synthetic oligonucleotide comprising a monkeypox virus sequence, a non-variola orthopox virus sequence, and an endogenous DNA or RNA sequence.

51. The method of claim 50, wherein the endogenous DNA or RNA sequence is a human RNaseP sequence.

52. The method of any one of claims 45-51, further comprising:
identifying the monkeypox virus as a variant or as reference form.

53. The method of any one of claims 45-52, wherein the detecting of the amplified target monkeypox virus nucleic acid sequence occurs during and/or after the subjecting of the reaction mixture to reaction conditions suitable to amplify the target monkeypox virus nucleic acid sequence.

54. The method of any one of claims 45-53, further including diagnosing a monkeypox infection in an organism from which the biological sample was obtained.

55. The method of claim 54, wherein the organism is a human subject.

56. The method of any one of claims 45-55, wherein the subjecting the reaction mixture to reaction conditions suitable to amplify the target monkeypox virus nucleic acid sequence includes performing a loop-mediated isothermal amplification (LAMP).

57. The method of any one of claims 45-56, wherein the method excludes purifying or extracting a nucleic-acid-containing portion from the biological sample.

58. The method of any one of claims 45-57, wherein the biological sample is an unpurified sample.

59. The method of any one of claims 45-58, further comprising:
heating the biological sample for a period of time sufficient to inactivate nucleases within the biological sample and/or to rupture eukaryotic cells, denature viral capsids, and/or disrupt a membrane portion of enveloped virions therein.

60. The method of any one of claims 57-59, further comprising:
heating the unpurified sample to a temperature at or above about 80° C., preferably at or above about 90° C., more preferably at or above about 95° C.

61. The method of claim 60, wherein the unpurified sample is heated for at least 5, 10, 15, 20, 25, 30, 35, or 40 minutes or for any range of time formed by an upper and lower bound selected therefrom.

62. The method of any one of claim 60 or 61, further comprising:
combining the heat-treated sample with a lysis buffer.

63. The method of claim 62, wherein the lysis buffer comprises a nucleic-acid-amenable buffer and a detergent and/or emulsifier.

64. The method of any one of claims 62-63, wherein the lysis buffer comprises a combination of TBE buffer and a polysorbate-type nonionic surfactant, such as Tween-20.

65. The method of any one of claims 45-64, wherein the biological sample comprises a pooled-subject sample.

66. The method of any one of claims 45-64, wherein detecting monkeypox virus within the biological sample occurs in less than about 3 hours from the time the biological sample is received, preferably less than about 2 hours.

67. The method of any one of claims 45-56 and 65-66, wherein the providing of the reaction mixture includes
heating the biological sample for 15-45 minutes, preferably about 30 minutes, at 95° C.;
mixing the heated biological sample with a lysis solution to form a volume of probative template;
creating a nucleic acid amplification reaction mixture comprising at least a portion of the volume of probative template, the composition according to claims 1-40, and a nucleic acid polymerase.

68. The method of any one of claims 45-67, further comprising:
receiving the biological sample.

69. The method of claim 68, wherein the receiving the biological sample comprises receiving a sample collection device or other container comprising the biological sample.

70. The method of claim 69, wherein the sample collection device is a sealable tube.

71. The method of any one of claims 68-71, wherein the biological sample is received following self-collection of the biological sample by a subject.

72. The method of any one of claims 68-71, wherein the receiving of the biological sample comprises receiving a raw saliva sample.

73. The method of any one of claims 67-71, further comprising:
equilibrating the heated biological sample to room temperature prior to mixing the heated sample with the lysis solution.

74. The method of any one of claims 67-74, wherein a sample collection device is used to receive and heat the biological sample.

75. A composition for multiplex detection of viral monkeypox nucleic acid and viral orthopox nucleic acid, the composition comprising:
a first primer-probe set including
a first forward primer and a first reverse primer each configured to hybridize to different ends of a first target nucleic acid sequence present in a target region of monkeypox viral DNA, to a DNA copy of the first target nucleic acid sequence, or to a complement of the first target nucleic acid sequence or a DNA copy of the complement, and
a first probe configured to hybridize to a first target nucleic acid subsequence that is complementary or identical to the first target nucleic acid sequence, or to a DNA copy of the first target nucleic acid subsequence,
wherein the first primer-probe set includes at least one nucleic acid sequence selected from SEQ ID NOs: 16-180;
a second primer-probe set including
a second forward primer and a second reverse primer each configured to hybridize to different ends of a second target sequence present in a target region of orthopox viral DNA, to a DNA copy of the second target nucleic acid sequence, or to a complement of the second target nucleic acid sequence or a DNA copy of the complement, and
a second probe configured to hybridize to a second target nucleic acid subsequence that is complementary or identical to the second target nucleic acid sequence, or to a DNA copy of the second target nucleic acid subsequence,
wherein the second primer-probe set includes at least one nucleic acid sequence selected from SEQ ID NOs: 4-12.

76. The composition according to claim 75, wherein
the first primer-probe set includes the at least one nucleic acid sequence selected from SEQ ID NOs: 41, 49, 55, 64, 96, 104, 110, 119, 151, 159, 165 and 174.

77. The composition of any one of claim 75 or 76 wherein, the first primer-probe set includes the at least one nucleic acid sequence having SEQ ID NO. 41.

78. The composition of any one of claims 75-77, wherein the first primer-probe set further includes the at least one nucleic acid sequence having SEQ ID NO. 96.

79. The composition of any one of claims 75-78, wherein the first primer-probe set further includes the at least one nucleic acid sequence having SEQ ID NO. 151.

80. The composition of any one of claim 75 or 76, wherein the first primer-probe set includes the at least one nucleic acid sequence having SEQ ID NO. 49.

81. The composition of any one of claim 75, 76 or 80, wherein the first primer-probe set further includes the at least one nucleic acid sequence having SEQ ID NO. 104.

82. The composition of any one of claim 75, 76, 80 or 81, wherein the first primer-probe set further includes the at least one nucleic acid sequence having SEQ ID NO. 159.

83. The composition of any one of claim 75 or 76, wherein the first primer-probe set includes the at least one nucleic acid sequence having SEQ ID NO. 55.

84. The composition of any one of claim 75, 76 or 83, wherein the first primer-probe set further includes the at least one nucleic acid sequence having SEQ ID NO. 110.

85. The composition of any one of claim 75, 76, 83 or 84, wherein the first primer-probe set further includes the at least one nucleic acid sequence having SEQ ID NO. 165.

86. The composition of any one of claim 75 or 76, wherein the first primer-probe set includes the at least one nucleic acid sequence having SEQ ID NO. 64.

87. The composition of any one of claim 75, 76 or 86, wherein the first primer-probe set further includes the at least one nucleic acid sequence having SEQ ID NO. 119.

88. The composition of any one of claim 75, 76, 86 or 87 wherein, the first primer-probe set further includes the at least one nucleic acid sequence having SEQ ID NO. 174.

89. The composition of any one of claims 75-88, wherein the second primer-probe set includes the at least one nucleic acid sequence having SEQ ID NOs. 4-6.

90. The composition of any one of claims 75-88, wherein the second primer-probe set includes the at least one nucleic acid sequence having SEQ ID NOs. 7-9.

91. The composition of any one of claims 75-88, wherein the second primer-probe set includes the at least one nucleic acid sequence having SEQ ID NOs. 10-12.

92. The composition according to any one of claims 75-91, further comprising:
a third primer-probe set including
a third forward primer and a third reverse primer each configured to hybridize to different ends of a third target nucleic acid sequence present in a target region of human ribonuclease P, and
a third probe configured to hybridize to a third target nucleic acid subsequence that is complementary or identical to the third target nucleic acid sequence, or to a DNA copy of the third target nucleic acid subsequence,
wherein the third primer-probe set includes at least one nucleic acid sequence selected from SEQ ID NOs: 13-15.

93. The composition of any one of claims 75-92, wherein the first nucleic acid probe, the second nucleic acid probe and the third nucleic acid probe each further comprises a different detectable label.

94. The composition of claim 93, wherein
the detectable label is a fluorescent label, and
the first nucleic acid probe, the second nucleic acid probe and the third nucleic acid probe each further comprises a different quencher that quenches the respective detectable label or the respective fluorescent label.

95. The composition of any one of claim 93 or 94, wherein the first nucleic acid probe, the second nucleic acid probe and the third nucleic acid probe are each labeled at a 5′ end with the detectable label, and at a 3′end with the quencher.

96. The composition of any one of claims 75-95, further comprising:
the biological sample, a polymerase, a buffer, and dNTPs.

97. The composition of any one of claims 75-96, wherein the biological sample is a nucleic acid sample, and the nucleic acid sample is a DNA sample.

98. The composition of any one of claims 75-96, wherein the biological sample is selected from the group consisting of a lesion fluid on a dry swab, a lesion fluid swab in viral transport media, lesion fluid on a slide, lesion crust, lesion roof, a saliva sample, a buccal sample, a nasal sample, a nasal pharyngeal sample, a blood sample, a urine sample, and a semen sample.

99. The composition of any one of claims 75-98, wherein the biological sample is a human sample.

100. The composition of any one of claims 75-98 wherein the biological sample is a non-human sample.


SEQUENCE LISTING (180 sequences)

SEQ ID NO: 1 - DNA, 136 bp, synthetic construct
  misc_feature 1..136: note = Synthetic
  source 1..136: mol_type = other DNA
ttcaggttttggtggaagtgtaactgctacttggtgggatactgaaggatatttcagagagttgtggatgttcgggttcgacatccaccgatggtgtcacgccactaatcggttcggtaacgtctgtggatggagg

SEQ ID NO: 2 - DNA, 151 bp, synthetic construct
  misc_feature 1..151: note = Synthetic
  source 1..151: mol_type = other DNA
tgtgcaactcttagccgaagcgtatgagtatagagcactatttctaaatcccatcagaccatatactgagttggctactatcttgtacgtatattgcatggaatcatagatggccttttcagttgaactggtagcctgttttaacatcttt

SEQ ID NO: 3 - DNA, 115 bp, synthetic construct
  misc_feature 1..115: note = Synthetic
  source 1..115: mol_type = other DNA
gggacttcagcatggcggtgtttgcagatttggacctgcgagcgggttctgacctgaaggctctgcgcggacttgtggagacagccgctcaccttggctattcagttgttgctat

SEQ ID NO: 4 - DNA, 25 bp, synthetic construct
  misc_feature 1..25: note = Synthetic
  source 1..25: mol_type = other DNA
gagtcggagccatctggtttaatat

SEQ ID NO: 5 - DNA, 25 bp, synthetic construct
  misc_feature 1..25: note = Synthetic
  source 1..25: mol_type = other DNA
ccacctttcactcaacaccttctta

SEQ ID NO: 6 - DNA, 18 bp, synthetic construct
  misc_feature 1..18: note = Synthetic
  source 1..18: mol_type = other DNA
aaccaccgataaatctag

SEQ ID NO: 7 - DNA, 25 bp, synthetic construct
  misc_feature 1..25: note = Synthetic
  source 1..25: mol_type = other DNA
agttccattagcctttccacttctg

SEQ ID NO: 8 - DNA, 25 bp, synthetic construct
  misc_feature 1..25: note = Synthetic
  source 1..25: mol_type = other DNA
ccaatgatcatcggtgagcctatta

SEQ ID NO: 9 - DNA, 20 bp, synthetic construct
  misc_feature 1..20: note = Synthetic
  source 1..20: mol_type = other DNA
ctggtaaagaaccaatttct

SEQ ID NO: 10 - DNA, 23 bp, synthetic construct
  misc_feature 1..23: note = Synthetic
  source 1..23: mol_type = other DNA
tcaactgaaaaggccatctatga

SEQ ID NO: 11 - DNA, 28 bp, synthetic construct
  misc_feature 1..28: note = Synthetic
  source 1..28: mol_type = other DNA
gagtatagagcactatttctaaatccca

SEQ ID NO: 12 - DNA, 32 bp, synthetic construct
  misc_feature 1..32: note = Synthetic
  source 1..32: mol_type = other DNA
ccatgcaatatacgtacaagatagtagccaac

SEQ ID NO: 13 - DNA, 19 bp, synthetic construct
  misc_feature 1..19: note = Synthetic
  source 1..19: mol_type = other DNA
agatttggacctgcgagcg

SEQ ID NO: 14 - DNA, 20 bp, synthetic construct
  misc_feature 1..20: note = Synthetic
  source 1..20: mol_type = other DNA
gagcggctgtctccacaagt

SEQ ID NO: 15 - DNA, 23 bp, synthetic construct
  misc_feature 1..23: note = Synthetic
  source 1..23: mol_type = other DNA
ttctgacctgaaggctctgcgcg

SEQ ID NO: 16 - DNA, 24 bp, synthetic construct
  misc_feature 1..24: note = Synthetic
  source 1..24: mol_type = other DNA
atgtgggatttggtagacctccta

SEQ ID NO: 17 - DNA, 19 bp, synthetic construct
  misc_feature 1..19: note = Synthetic
  source 1..19: mol_type = other DNA
gcgctcgatgggacatttc

SEQ ID NO: 18 - DNA, 25 bp, synthetic construct
  misc_feature 1..25: note = Synthetic
  source 1..25: mol_type = other DNA
gcaagcagccagtatagaaattgtt

SEQ ID NO: 19 - DNA, 19 bp, synthetic construct
  misc_feature 1..19: note = Synthetic
  source 1..19: mol_type = other DNA
acagaagcagctgcagcaa

SEQ ID NO: 20 - DNA, 24 bp, synthetic construct
  misc_feature 1..24: note = Synthetic
  source 1..24: mol_type = other DNA
tcggttttggtgaaatccaaggat

SEQ ID NO: 21 - DNA, 26 bp, synthetic construct
  misc_feature 1..26: note = Synthetic
  source 1..26: mol_type = other DNA
cgtaagaacacgatggagagtaatcg

SEQ ID NO: 22 - DNA, 25 bp, synthetic construct
  misc_feature 1..25: note = Synthetic
  source 1..25: mol_type = other DNA
cgagttcccatagctggttcaaaat

SEQ ID NO: 23 - DNA, 22 bp, synthetic construct
  misc_feature 1..22: note = Synthetic
  source 1..22: mol_type = other DNA
cgatggctcttgaaaccaaggt

SEQ ID NO: 24 - DNA, 26 bp, synthetic construct
  misc_feature 1..26: note = Synthetic
  source 1..26: mol_type = other DNA
ggcatcttgaatagaaacaacaccat

SEQ ID NO: 25 - DNA, 25 bp, synthetic construct
  misc_feature 1..25: note = Synthetic
  source 1..25: mol_type = other DNA
cgttcgattaacccaactcatccat

SEQ ID NO: 26 - DNA, 21 bp, synthetic construct
  misc_feature 1..21: note = Synthetic
  source 1..21: mol_type = other DNA
cgggagagtccttaccttgtc

SEQ ID NO: 27 - DNA, 24 bp, synthetic construct
  misc_feature 1..24: note = Synthetic
  source 1..24: mol_type = other DNA
tcataggtctagccacagaaacca

SEQ ID NO: 28 - DNA, 24 bp, synthetic construct
  misc_feature 1..24: note = Synthetic
  source 1..24: mol_type = other DNA
gttcggtgcagctagtactcttaa

SEQ ID NO: 29 - DNA, 25 bp, synthetic construct
  misc_feature 1..25: note = Synthetic
  source 1..25: mol_type = other DNA
ctcacctgtcatgtctggtaattca

SEQ ID NO: 30 - DNA, 25 bp, synthetic construct
  misc_feature 1..25: note = Synthetic
  source 1..25: mol_type = other DNA
cgttagtaaaacatggcgaggaaat

SEQ ID NO: 31 - DNA, 27 bp, synthetic construct
  misc_feature 1..27: note = Synthetic
  source 1..27: mol_type = other DNA
gatcctccttgtatttgtcacattcct

SEQ ID NO: 32 - DNA, 22 bp, synthetic construct
  misc_feature 1..22: note = Synthetic
  source 1..22: mol_type = other DNA
cctgctcggttacttctgtgta

SEQ ID NO: 33 - DNA, 26 bp, synthetic construct
  misc_feature 1..26: note = Synthetic
  source 1..26: mol_type = other DNA
accacagactgaaagtatctctacct

SEQ ID NO: 34 - DNA, 25 bp, synthetic construct
  misc_feature 1..25: note = Synthetic
  source 1..25: mol_type = other DNA
gcattagtaagtgctgcaaaacaga

SEQ ID NO: 35 - DNA, 25 bp, synthetic construct
  misc_feature 1..25: note = Synthetic
  source 1..25: mol_type = other DNA
gccgtgggataatgttaaggattgt

SEQ ID NO: 36 - DNA, 27 bp, synthetic construct
  misc_feature 1..27: note = Synthetic
  source 1..27: mol_type = other DNA
aggcgtttatcatatctgtgatttcgt

SEQ ID NO: 37 - DNA, 23 bp, synthetic construct
  misc_feature 1..23: note = Synthetic
  source 1..23: mol_type = other DNA
gggtgaagttattcccctcatca

SEQ ID NO: 38 - DNA, 22 bp, synthetic construct
  misc_feature 1..22: note = Synthetic
  source 1..22: mol_type = other DNA
actccctgaataccgtgactga

SEQ ID NO: 39 - DNA, 25 bp, synthetic construct
  misc_feature 1..25: note = Synthetic
  source 1..25: mol_type = other DNA
cgtataatgccgtatccccaaaagt

SEQ ID NO: 40 - DNA, 21 bp, synthetic construct
  misc_feature 1..21: note = Synthetic
  source 1..21: mol_type = other DNA
gcgatgacagatcccctatcc

SEQ ID NO: 41 - DNA, 25 bp, synthetic construct
  misc_feature 1..25: note = Synthetic
  source 1..25: mol_type = other DNA
ctattgtgtcacctctcaccagaaa

SEQ ID NO: 42 - DNA, 25 bp, synthetic construct
  misc_feature 1..25: note = Synthetic
  source 1..25: mol_type = other DNA
gcattgatgacactggatgatttgg

SEQ ID NO: 43 - DNA, 26 bp, synthetic construct
  misc_feature 1..26: note = Synthetic
  source 1..26: mol_type = other DNA
gggtaaactgtctccaatttttgtgt

SEQ ID NO: 44 - DNA, 21 bp, synthetic construct
  misc_feature 1..21: note = Synthetic
  source 1..21: mol_type = other DNA
cggaatcgcaaaccacttgtg

SEQ ID NO: 45 - DNA, 24 bp, synthetic construct
  misc_feature 1..24: note = Synthetic
  source 1..24: mol_type = other DNA
gaagagtatacagaagcagctgca

SEQ ID NO: 46 - DNA, 23 bp, synthetic construct
  misc_feature 1..23: note = Synthetic
  source 1..23: mol_type = other DNA
tgaatgatatggccgctctagga

SEQ ID NO: 47 - DNA, 28 bp, synthetic construct
  misc_feature 1..28: note = Synthetic
  source 1..28: mol_type = other DNA
ggacaaatctggaaaaacaacacaatgt

SEQ ID NO: 48 - DNA, 24 bp, synthetic construct
  misc_feature 1..24: note = Synthetic
  source 1..24: mol_type = other DNA
ggtcctccttatctcctccagtag

SEQ ID NO: 49 - DNA, 24 bp, synthetic construct
  misc_feature 1..24: note = Synthetic
  source 1..24: mol_type = other DNA
gctacttggtgggatactgaagga

SEQ ID NO: 50 - DNA, 23 bp, synthetic construct
  misc_feature 1..23: note = Synthetic
  source 1..23: mol_type = other DNA
cggtacacgtaccagcatttgta

SEQ ID NO: 51 - DNA, 24 bp, synthetic construct
  misc_feature 1..24: note = Synthetic
  source 1..24: mol_type = other DNA
ccaatgcgtccatgttgtctattt

SEQ ID NO: 52 - DNA, 25 bp, synthetic construct
  misc_feature 1..25: note = Synthetic
  source 1..25: mol_type = other DNA
cgagcgtgaataacttacagatgga

SEQ ID NO: 53 - DNA, 25 bp, synthetic construct
  misc_feature 1..25: note = Synthetic
  source 1..25: mol_type = other DNA
ccgcaatactcctcattacgtttca

SEQ ID NO: 54 - DNA, 27 bp, synthetic construct
  misc_feature 1..27: note = Synthetic
  source 1..27: mol_type = other DNA
gcagaaataaccgaattagtcgtcgaa

SEQ ID NO: 55 - DNA, 25 bp, synthetic construct
  misc_feature 1..25: note = Synthetic
  source 1..25: mol_type = other DNA
ggttcggatacctttacatctcaca

SEQ ID NO: 56 - DNA, 24 bp, synthetic construct
  misc_feature 1..24: note = Synthetic
  source 1..24: mol_type = other DNA
cgtgcgattcagaacacacttttc

SEQ ID NO: 57 - DNA, 25 bp, synthetic construct
  misc_feature 1..25: note = Synthetic
  source 1..25: mol_type = other DNA
caacaggtcgctatatccaccaata

SEQ ID NO: 58 - DNA, 23 bp, synthetic construct
  misc_feature 1..23: note = Synthetic
  source 1..23: mol_type = other DNA
gtccttccaccagcatctagttg

SEQ ID NO: 59 - DNA, 25 bp, synthetic construct
  misc_feature 1..25: note = Synthetic
  source 1..25: mol_type = other DNA
tcggattttgtgggttcttgatctt

SEQ ID NO: 60 - DNA, 23 bp, synthetic construct
  misc_feature 1..23: note = Synthetic
  source 1..23: mol_type = other DNA
gtgtttcgaatgcctcgtacaag

SEQ ID NO: 61 - DNA, 24 bp, synthetic construct
  misc_feature 1..24: note = Synthetic
  source 1..24: mol_type = other DNA
catattctcacaccgtctcttcca

SEQ ID NO: 62 - DNA, 25 bp, synthetic construct
  misc_feature 1..25: note = Synthetic
  source 1..25: mol_type = other DNA
ggcataatccggatgttgtgtagta

SEQ ID NO: 63 - DNA, 30 bp, synthetic construct
  misc_feature 1..30: note = Synthetic
  source 1..30: mol_type = other DNA
agaaagttacgtttacatgtgattcaggat

SEQ ID NO: 64 - DNA, 20 bp, synthetic construct
  misc_feature 1..20: note = Synthetic
  source 1..20: mol_type = other DNA
gtttgctgctaatcgcgaca

SEQ ID NO: 65 - DNA, 28 bp, synthetic construct
  misc_feature 1..28: note = Synthetic
  source 1..28: mol_type = other DNA
ctcaacaagttctcaataccccaataga

SEQ ID NO: 66 - DNA, 24 bp, synthetic construct
  misc_feature 1..24: note = Synthetic
  source 1..24: mol_type = other DNA
gctggcgaccatcatattatctct

SEQ ID NO: 67 - DNA, 28 bp, synthetic construct
  misc_feature 1..28: note = Synthetic
  source 1..28: mol_type = other DNA
caagatcattctacgtcctatggatgtg

SEQ ID NO: 68 - DNA, 25 bp, synthetic construct
  misc_feature 1..25: note = Synthetic
  source 1..25: mol_type = other DNA
gacacgctggacaatctagtattca

SEQ ID NO: 69 - DNA, 24 bp, synthetic construct
  misc_feature 1..24: note = Synthetic
  source 1..24: mol_type = other DNA
cgacggtacaacattccaaagatc

SEQ ID NO: 70 - DNA, 27 bp, synthetic construct
  misc_feature 1..27: note = Synthetic
  source 1..27: mol_type = other DNA
tgtggttaaatgggaaaggctagaaaa

SEQ ID NO: 71 - DNA, 23 bp, synthetic construct
  misc_feature 1..23: note = Synthetic
  source 1..23: mol_type = other DNA
tgaatagccccagactgtatgct

SEQ ID NO: 72 - DNA, 25 bp, synthetic construct
  misc_feature 1..25: note = Synthetic
  source 1..25: mol_type = other DNA
acactgcagttagttttaccaccat

SEQ ID NO: 73 - DNA, 24 bp, synthetic construct
  misc_feature 1..24: note = Synthetic
  source 1..24: mol_type = other DNA
gtggtacagcacgtagtgactatt

SEQ ID NO: 74 - DNA, 22 bp, synthetic construct
  misc_feature 1..22: note = Synthetic
  source 1..22: mol_type = other DNA
cggatgatctgcacagaactca

SEQ ID NO: 75 - DNA, 25 bp, synthetic construct
  misc_feature 1..25: note = Synthetic
  source 1..25: mol_type = other DNA
cagcgcatctatctaactacatcgt

SEQ ID NO: 76 - DNA, 24 bp, synthetic construct
  misc_feature 1..24: note = Synthetic
  source 1..24: mol_type = other DNA
cgtgttggctgcaaaatatatcca

SEQ ID NO: 77 - DNA, 22 bp, synthetic construct
  misc_feature 1..22: note = Synthetic
  source 1..22: mol_type = other DNA
tgcgacgttgtatacgccataa

SEQ ID NO: 78 - DNA, 28 bp, synthetic construct
  misc_feature 1..28: note = Synthetic
  source 1..28: mol_type = other DNA
ctaatgaaaaagacttctcaccgatcga

SEQ ID NO: 79 - DNA, 22 bp, synthetic construct
  misc_feature 1..22: note = Synthetic
  source 1..22: mol_type = other DNA
ccgactggttatccggaatctg

SEQ ID NO: 80 - DNA, 24 bp, synthetic construct
  misc_feature 1..24: note = Synthetic
  source 1..24: mol_type = other DNA
gtgcaaccgatgatacttcactca

SEQ ID NO: 81 - DNA, 25 bp, synthetic construct
  misc_feature 1..25: note = Synthetic
  source 1..25: mol_type = other DNA
gaatcctctgatggaaacactgtga

SEQ ID NO: 82 - DNA, 25 bp, synthetic construct
  misc_feature 1..25: note = Synthetic
  source 1..25: mol_type = other DNA
ctggatgctctcaagttatgacaga

SEQ ID NO: 83 - DNA, 25 bp, synthetic construct
  misc_feature 1..25: note = Synthetic
  source 1..25: mol_type = other DNA
gactttttatgaagagccgcgttta

SEQ ID NO: 84 - DNA, 22 bp, synthetic construct
  misc_feature 1..22: note = Synthetic
  source 1..22: mol_type = other DNA
gcaatccgaatgtgtccatcca

SEQ ID NO: 85 - DNA, 17 bp, synthetic construct
  misc_feature 1..17: note = Synthetic
  source 1..17: mol_type = other DNA
tccgccgatgtgaacgt

SEQ ID NO: 86 - DNA, 24 bp, synthetic construct
  misc_feature 1..24: note = Synthetic
  source 1..24: mol_type = other DNA
gtcgtaatgtcgaatgggaactgt

SEQ ID NO: 87 - DNA, 26 bp, synthetic construct
  misc_feature 1..26: note = Synthetic
  source 1..26: mol_type = other DNA
gcatgcttcccatattctacataggt

SEQ ID NO: 88 - DNA, 29 bp, synthetic construct
  misc_feature 1..29: note = Synthetic
  source 1..29: mol_type = other DNA
ggctattatcttctttacgtagtgatcgt

SEQ ID NO: 89 - DNA, 25 bp, synthetic construct
  misc_feature 1..25: note = Synthetic
  source 1..25: mol_type = other DNA
ggtgcatattgcaaactggtcatta

SEQ ID NO: 90 - DNA, 24 bp, synthetic construct
  misc_feature 1..24: note = Synthetic
  source 1..24: mol_type = other DNA
ggccgaaatctctctgatggtttt

SEQ ID NO: 91 - DNA, 24 bp, synthetic construct
  misc_feature 1..24: note = Synthetic
  source 1..24: mol_type = other DNA
acgtgtaccgtatccataggtgat

SEQ ID NO: 92 - DNA, 25 bp, synthetic construct
  misc_feature 1..25: note = Synthetic
  source 1..25: mol_type = other DNA
ccatagcatacgtgttctcatccat

SEQ ID NO: 93 - DNA, 24 bp, synthetic construct
  misc_feature 1..24: note = Synthetic
  source 1..24: mol_type = other DNA
cgctttctctatgggtccatctat

SEQ ID NO: 94 - DNA, 25 bp, synthetic construct
  misc_feature 1..25: note = Synthetic
  source 1..25: mol_type = other DNA
gctccaaacatatggatgcgagata

SEQ ID NO: 95 - DNA, 24 bp, synthetic construct
  misc_feature 1..24: note = Synthetic
  source 1..24: mol_type = other DNA
gcaaatgtcaacgcagtaatgagt

SEQ ID NO: 96 - DNA, 24 bp, synthetic construct
  misc_feature 1..24: note = Synthetic
  source 1..24: mol_type = other DNA
gaccatcatttgtagggcgtctag

SEQ ID NO: 97 - DNA, 25 bp, synthetic construct
  misc_feature 1..25: note = Synthetic
  source 1..25: mol_type = other DNA
cccgaatcggagtctactttaagtt

SEQ ID NO: 98 - DNA, 25 bp, synthetic construct
  misc_feature 1..25: note = Synthetic
  source 1..25: mol_type = other DNA
cacgcataccttttcaatgagcatt

SEQ ID NO: 99 - DNA, 26 bp, synthetic construct
  misc_feature 1..26: note = Synthetic
  source 1..26: mol_type = other DNA
acgggaatgtactatctacgtacgaa

SEQ ID NO: 100 - DNA, 22 bp, synthetic construct
  misc_feature 1..22: note = Synthetic
  source 1..22: mol_type = other DNA
cggatgatctgcacagaactca

SEQ ID NO: 101 - DNA, 26 bp, synthetic construct
  misc_feature 1..26: note = Synthetic
  source 1..26: mol_type = other DNA
gcctttcccatttaaccacatagtct

SEQ ID NO: 102 - DNA, 25 bp, synthetic construct
  misc_feature 1..25: note = Synthetic
  source 1..25: mol_type = other DNA
gtggatcgctgaggaaagttaagat

SEQ ID NO: 103 - DNA, 20 bp, synthetic construct
  misc_feature 1..20: note = Synthetic
  source 1..20: mol_type = other DNA
ccgcgtacgacaagcatttg

SEQ ID NO: 104 - DNA, 21 bp, synthetic construct
  misc_feature 1..21: note = Synthetic
  source 1..21: mol_type = other DNA
cgattagtggcgtgacaccat

SEQ ID NO: 105 - DNA, 27 bp, synthetic construct
  misc_feature 1..27: note = Synthetic
  source 1..27: mol_type = other DNA
cgagagtggagatattacaggagaaga

SEQ ID NO: 106 - DNA, 25 bp, synthetic construct
  misc_feature 1..25: note = Synthetic
  source 1..25: mol_type = other DNA
actggtggtagaactgttcctagaa

SEQ ID NO: 107 - DNA, 24 bp, synthetic construct
  misc_feature 1..24: note = Synthetic
  source 1..24: mol_type = other DNA
ttttgctctattgacgggaacagt

SEQ ID NO: 108 - DNA, 27 bp, synthetic construct
  misc_feature 1..27: note = Synthetic
  source 1..27: mol_type = other DNA
tgcgcttctcaattggataatctatgt

SEQ ID NO: 109 - DNA, 24 bp, synthetic construct
  misc_feature 1..24: note = Synthetic
  source 1..24: mol_type = other DNA
ctcgtcctcaatgaatgtctccat

SEQ ID NO: 110 - DNA, 25 bp, synthetic construct
  misc_feature 1..25: note = Synthetic
  source 1..25: mol_type = other DNA
cgatcgcgtctctacctgattacta

SEQ ID NO: 111 - DNA, 25 bp, synthetic construct
  misc_feature 1..25: note = Synthetic
  source 1..25: mol_type = other DNA
gtttgactttatcgtacgcgtcatt

SEQ ID NO: 112 - DNA, 24 bp, synthetic construct
  misc_feature 1..24: note = Synthetic
  source 1..24: mol_type = other DNA
cccgaaaatgaattgcgtgactat

SEQ ID NO: 113 - DNA, 25 bp, synthetic construct
  misc_feature 1..25: note = Synthetic
  source 1..25: mol_type = other DNA
gattccttgtagatgaacccacagt

SEQ ID NO: 114 - DNA, 26 bp, synthetic construct
  misc_feature 1..26: note = Synthetic
  source 1..26: mol_type = other DNA
tgttgatcggtatttgtgtagcagtt

SEQ ID NO: 115 - DNA, 26 bp, synthetic construct
  misc_feature 1..26: note = Synthetic
  source 1..26: mol_type = other DNA
cgcaattctaaatcgttggtcaaaga

SEQ ID NO: 116 - DNA, 25 bp, synthetic construct
  misc_feature 1..25: note = Synthetic
  source 1..25: mol_type = other DNA
gtgtcgttgactggatacaggttaa

SEQ ID NO: 117 - DNA, 25 bp, synthetic construct
  misc_feature 1..25: note = Synthetic
  source 1..25: mol_type = other DNA
ggagtaacaacggtagaattggaca

SEQ ID NO: 118 - DNA, 25 bp, synthetic construct
  misc_feature 1..25: note = Synthetic
  source 1..25: mol_type = other DNA
gcacattttcttgcatggattttcg

SEQ ID NO: 119 - DNA, 23 bp, synthetic construct
  misc_feature 1..23: note = Synthetic
  source 1..23: mol_type = other DNA
cccgggagacacggatttattaa

SEQ ID NO: 120 - DNA, 25 bp, synthetic construct
  misc_feature 1..25: note = Synthetic
  source 1..25: mol_type = other DNA
gtggatatagcgacctgttggaaat

SEQ ID NO: 121 - DNA, 29 bp, synthetic construct
  misc_feature 1..29: note = Synthetic
  source 1..29: mol_type = other DNA
gatgaagcgattgaaataggactaaagtc

SEQ ID NO: 122 - DNA, 27 bp, synthetic construct
  misc_feature 1..27: note = Synthetic
  source 1..27: mol_type = other DNA
gtagccaactcagtatatggtctgatg

SEQ ID NO: 123 - DNA, 24 bp, synthetic construct
  misc_feature 1..24: note = Synthetic
  source 1..24: mol_type = other DNA
tggattcaccaaatgcccaaagat

SEQ ID NO: 124 - DNA, 24 bp, synthetic construct
  misc_feature 1..24: note = Synthetic
  source 1..24: mol_type = other DNA
agctgagaaggaaggaccatatga

SEQ ID NO: 125 - DNA, 26 bp, synthetic construct
  misc_feature 1..26: note = Synthetic
  source 1..26: mol_type = other DNA
ccataaatcaccatgtttgacacgtt

SEQ ID NO: 126 - DNA, 17 bp, synthetic construct
  misc_feature 1..17: note = Synthetic
  source 1..17: mol_type = other DNA
ttgttagcgggtatccc

SEQ ID NO: 127 - DNA, 20 bp, synthetic construct
  misc_feature 1..20: note = Synthetic
  source 1..20: mol_type = other DNA
aacgtagaccgtttaataat

SEQ ID NO: 128 - DNA, 16 bp, synthetic construct
  misc_feature 1..16: note = Synthetic
  source 1..16: mol_type = other DNA
ctcccatggaatgacc

SEQ ID NO: 129 - DNA, 19 bp, synthetic construct
  misc_feature 1..19: note = Synthetic
  source 1..19: mol_type = other DNA
ctgttgatgcacagtctgc

SEQ ID NO: 130 - DNA, 18 bp, synthetic construct
  misc_feature 1..18: note = Synthetic
  source 1..18: mol_type = other DNA
caatccacagacaaaaca

SEQ ID NO: 131 - DNA, 17 bp, synthetic construct
  misc_feature 1..17: note = Synthetic
  source 1..17: mol_type = other DNA
ccgggatctgcgattaa

SEQ ID NO: 132 - DNA, 19 bp, synthetic construct
  misc_feature 1..19: note = Synthetic
  source 1..19: mol_type = other DNA
atgtaacttgagaaaatcc

SEQ ID NO: 133 - DNA, 18 bp, synthetic construct
  misc_feature 1..18: note = Synthetic
  source 1..18: mol_type = other DNA
ttccaaccggactcattg

SEQ ID NO: 134 - DNA, 18 bp, synthetic construct
  misc_feature 1..18: note = Synthetic
  source 1..18: mol_type = other DNA
ccgagatcgtagaggttc

SEQ ID NO: 135 - DNA, 21 bp, synthetic construct
  misc_feature 1..21: note = Synthetic
  source 1..21: mol_type = other DNA
atgtgtctgaatcgataactc

SEQ ID NO: 136 - DNA, 16 bp, synthetic construct
  misc_feature 1..16: note = Synthetic
  source 1..16: mol_type = other DNA
cacgctggacaatcta

SEQ ID NO: 137 - DNA, 22 bp, synthetic construct
  misc_feature 1..22: note = Synthetic
  source 1..22: mol_type = other DNA
acgtaggtctaaatccaactat

SEQ ID NO: 138 - DNA, 21 bp, synthetic construct
  misc_feature 1..21: note = Synthetic
  source 1..21: mol_type = other DNA
acgttcggagataataaagca

SEQ ID NO: 139 - DNA, 18 bp, synthetic construct
  misc_feature 1..18: note = Synthetic
  source 1..18: mol_type = other DNA
ctgttgaagcatttcttg

SEQ ID NO: 140 - DNA, 20 bp, synthetic construct
  misc_feature 1..20: note = Synthetic
  source 1..20: mol_type = other DNA
cttgactttttcacaaactc

SEQ ID NO: 141 - DNA, 20 bp, synthetic construct
  misc_feature 1..20: note = Synthetic
  source 1..20: mol_type = other DNA
ccagacttcgtatggatatc

SEQ ID NO: 142 - DNA, 19 bp, synthetic construct
  misc_feature 1..19: note = Synthetic
  source 1..19: mol_type = other DNA
atacactgtgtcaaaactg

SEQ ID NO: 143 - DNA, 17 bp, synthetic construct
  misc_feature 1..17: note = Synthetic
  source 1..17: mol_type = other DNA
aagatgtcggagttctc

SEQ ID NO: 144 - DNA, 21 bp, synthetic construct
  misc_feature 1..21: note = Synthetic
  source 1..21: mol_type = other DNA
caaagtcgcagtattgttaac

SEQ ID NO: 145 - DNA, 18 bp, synthetic construct
  misc_feature 1..18: note = Synthetic
  source 1..18: mol_type = other DNA
aacaacggatgatctctg

SEQ ID NO: 146 - DNA, 20 bp, synthetic construct
  misc_feature 1..20: note = Synthetic
  source 1..20: mol_type = other DNA
actgggttgtaatgttatcc

SEQ ID NO: 147 - DNA, 16 bp, synthetic construct
  misc_feature 1..16: note = Synthetic
  source 1..16: mol_type = other DNA
ttggtcagaaattttg

SEQ ID NO: 148 - DNA, 16 bp, synthetic construct
  misc_feature 1..16: note = Synthetic
  source 1..16: mol_type = other DNA
atcctgtgggtttctc

SEQ ID NO: 149 - DNA, 21 bp, synthetic construct
  misc_feature 1..21: note = Synthetic
  source 1..21: mol_type = other DNA
aataatcaggacgaataattc

SEQ ID NO: 150 - DNA, 20 bp, synthetic construct
  misc_feature 1..20: note = Synthetic
  source 1..20: mol_type = other DNA
cacgtcaagacatgtatatc

SEQ ID NO: 151 - DNA, 19 bp, synthetic construct
  misc_feature 1..19: note = Synthetic
  source 1..19: mol_type = other DNA
tcgtcgtctagtagctcct

SEQ ID NO: 152 - DNA, 21 bp, synthetic construct
  misc_feature 1..21: note = Synthetic
  source 1..21: mol_type = other DNA
acagtatggagacattgatct

SEQ ID NO: 153 - DNA, 15 bp, synthetic construct
  misc_feature 1..15: note = Synthetic
  source 1..15: mol_type = other DNA
cagtggcactaatcc

SEQ ID NO: 154 - DNA, 21 bp, synthetic construct
  misc_feature 1..21: note = Synthetic
  source 1..21: mol_type = other DNA
ccgctcccattcaattcacat

SEQ ID NO: 155 - DNA, 19 bp, synthetic construct
  misc_feature 1..19: note = Synthetic
  source 1..19: mol_type = other DNA
ccactagtacagaagttgc

SEQ ID NO: 156 - DNA, 20 bp, synthetic construct
  misc_feature 1..20: note = Synthetic
  source 1..20: mol_type = other DNA
ttcaataggaggacactttg

SEQ ID NO: 157 - DNA, 17 bp, synthetic construct
  misc_feature 1..17: note = Synthetic
  source 1..17: mol_type = other DNA
ccggcaaacacgataaa

SEQ ID NO: 158 - DNA, 19 bp, synthetic construct
  misc_feature 1..19: note = Synthetic
  source 1..19: mol_type = other DNA
catgtggttcttcaatacc

SEQ ID NO: 159 - DNA, 17 bp, synthetic construct
  misc_feature 1..17: note = Synthetic
  source 1..17: mol_type = other DNA
tcgggttcgacatccac

SEQ ID NO: 160 - DNA, 15 bp, synthetic construct
  misc_feature 1..15: note = Synthetic
  source 1..15: mol_type = other DNA
ttctgcgcaatgcca

SEQ ID NO: 161 - DNA, 18 bp, synthetic construct
  misc_feature 1..18: note = Synthetic
  source 1..18: mol_type = other DNA
caacaggtcgctatatcc

SEQ ID NO: 162 - DNA, 21 bp, synthetic construct
  misc_feature 1..21: note = Synthetic
  source 1..21: mol_type = other DNA
aagcaatagtgatagattttc

SEQ ID NO: 163 - DNA, 19 bp, synthetic construct
  misc_feature 1..19: note = Synthetic
  source 1..19: mol_type = other DNA
aacaaaatgggcattctag

SEQ ID NO: 164 - DNA, 20 bp, synthetic construct
  misc_feature 1..20: note = Synthetic
  source 1..20: mol_type = other DNA
ccccattcctttacaatgac

SEQ ID NO: 165 - DNA, 21 bp, synthetic construct
  misc_feature 1..21: note = Synthetic
  source 1..21: mol_type = other DNA
acttagacaagcctgtaaatg

SEQ ID NO: 166 - DNA, 19 bp, synthetic construct
  misc_feature 1..19: note = Synthetic
  source 1..19: mol_type = other DNA
cctctctaccagaaggttg

SEQ ID NO: 167 - DNA, 20 bp, synthetic construct
  misc_feature 1..20: note = Synthetic
  source 1..20: mol_type = other DNA
cagttctaccaccagtaatt

SEQ ID NO: 168 - DNA, 22 bp, synthetic construct
  misc_feature 1..22: note = Synthetic
  source 1..22: mol_type = other DNA
aagctttgtatcccatatattg

SEQ ID NO: 169 - DNA, 20 bp, synthetic construct
  misc_feature 1..20: note = Synthetic
  source 1..20: mol_type = other DNA
tcgccatcctatacacgctg

SEQ ID NO: 170 - DNA, 19 bp, synthetic construct
  misc_feature 1..19: note = Synthetic
  source 1..19: mol_type = other DNA
ctatggcatccttgaaatc

SEQ ID NO: 171 - DNA, 17 bp, synthetic construct
  misc_feature 1..17: note = Synthetic
  source 1..17: mol_type = other DNA
tacgggttcgcatttat

SEQ ID NO: 172 - DNA, 16 bp, synthetic construct
  misc_feature 1..16: note = Synthetic
  source 1..16: mol_type = other DNA
tctggcagccgaaata

SEQ ID NO: 173 - DNA, 16 bp, synthetic construct
  misc_feature 1..16: note = Synthetic
  source 1..16: mol_type = other DNA
tcgcagacagcatttg

SEQ ID NO: 174 - DNA, 18 bp, synthetic construct
  misc_feature 1..18: note = Synthetic
  source 1..18: mol_type = other DNA
acgtaacgtcaagacttt

SEQ ID NO: 175 - DNA, 17 bp, synthetic construct
  misc_feature 1..17: note = Synthetic
  source 1..17: mol_type = other DNA
cacccaatgcgtccatg

SEQ ID NO: 176 - DNA, 18 bp, synthetic construct
  misc_feature 1..18: note = Synthetic
  source 1..18: mol_type = other DNA
ttctcgcaactgtctttg

SEQ ID NO: 177 - DNA, 17 bp, synthetic construct
  misc_feature 1..17: note = Synthetic
  source 1..17: mol_type = other DNA
acgcttcggctaagagt

SEQ ID NO: 178 - DNA, 18 bp, synthetic construct
  misc_feature 1..18: note = Synthetic
  source 1..18: mol_type = other DNA
cctctgatggaaacactg

SEQ ID NO: 179 - DNA, 18 bp, synthetic construct
  misc_feature 1..18: note = Synthetic
  source 1..18: mol_type = other DNA
acgccagtaacggtattc

SEQ ID NO: 180 - DNA, 16 bp, synthetic construct
  misc_feature 1..16: note = Synthetic
  source 1..16: mol_type = other DNA
ctgtcgccgtctattc